(19) United States
(12) Patent Application Publication
(10) Pub. No.: US 2015/0191541 A1
(43) Pub. Date: Jul. 9, 2015

(54) ANTIBODIES THAT RECOGNIZE IAPP

(71) Applicant: Neotope Biosciences Limited, Dublin (IE)

(72) Inventors: Robin Barbour, Walnut Creek, CA (US); Tarlochan S. Nijjar, Orinda, CA (US)

(21) Appl. No.: 14/561,609

(22) Filed: Dec. 5, 2014

Related U.S. Application Data
(60) Provisional application No. 61/913,184, filed on Dec. 6, 2013.
(60) Provisional application No. 62/014,033, filed on Jun. 18, 2014.
(60) Provisional application No. 62/023,622, filed on Jul. 11, 2014.

(51) Int. Cl. C07K 16/26; G01N 33/74

(52) U.S. Cl. CPC C07K 16/26; G01N 33/74; C07K 2317/565; C07K 2317/55; A61K 2039/505

(57) ABSTRACT

The invention provides monoclonal antibody 8A9 and related antibodies. The 8A9 antibody binds to an epitope within residues 1-10 of IAPP. The antibodies of the invention are useful, for example, for treating disorders associated with IAPP accumulation, particularly accumulation of IAPP deposits. Such disorders include type 2 diabetes, metabolic syndrome, impaired insulin tolerance, impaired glucose tolerance, insulinomas, and related conditions.

### CROSS-REFERENCES TO RELATED APPLICATIONS

This application claims the benefit under 35 U.S.C. §119(e) of U.S. Application No. 61/913,184, filed Dec. 6, 2013; U.S. Application No. 62/014,033, filed Jun. 18, 2014; and U.S. Application No. 62/023,622, filed Jul. 11, 2014. Each of the aforementioned applications is incorporated in its entirety herein for all purposes.

### REFERENCE TO A SEQUENCE LISTING

The Sequence Listing written in file 451803SEQLIST.txt is 42.8 kilobytes, was created on Dec. 4, 2014, and is hereby incorporated by reference.

### BACKGROUND

Several diseases are thought to be caused by the abnormal folding and aggregation of disease-specific proteins. These proteins can accumulate into pathologically diagnostic accumulations, known as amyloids, which are visualized by certain histologic stains. Amyloids are thought to elicit inflammatory responses and have multiple negative consequences for the involved tissues. In addition, smaller aggregates of abnormally folded protein may exist and exert cytotoxic effects.

Type-2 diabetes (T2D) is a common disease where amyloid accumulations are often seen in the pancreas. The amyloid accumulations include islet-amyloid polypeptide (IAPP), also known as amylin. Accumulating evidence associates toxic IAPP oligomers and IAPP deposits with T2D. See, e.g., Haataja et al. (2008), Islet amyloid in type 2 diabetes, and the toxic oligomer hypothesis, Endocrine Reviews 29:303-316. For example, there is evidence of the involvement of toxic IAPP oligomers in Beta cell apoptosis in T2D. See Janson et al. (1999), The mechanism of islet amyloid polypeptide toxicity is membrane disruption by intermediate-sized toxic amyloid particles, Diabetes 48:491-498; Lorenzo et al. (1994), Pancreatic islet cell toxicity of amylin associated with type-2 diabetes mellitus, Nature 368:756-760; Ritzel & Butler (2003), Replication increases beta-cell vulnerability to human islet amyloid polypeptide-induced apoptosis, Diabetes 52:1701-1708. In addition, humans, monkeys and cats express an amyloidogenic toxic form of IAPP and develop diabetes characterized by islet amyloid deposits. See O'Brien et al. (1993), Islet amyloid polypeptide: A review of its biology and potential roles in the pathogenesis of diabetes mellitus, Vet. Pathol. 30:317-332. IAPP deposits are also found in insulinomas. See O'Brien et al. (1994), Islet amyloid polypeptide in human insulinomas. Evidence for intracellular amyloidogenesis, Diabetes 43: 329-336.

Pre-diabetes is a condition in which individuals have blood glucose levels higher than normal but not high enough to be classified as diabetes. People with pre-diabetes have an increased risk of developing T2D. People with pre-diabetes have impaired fasting glucose (“IFG”) or impaired glucose tolerance (“IGT”), and some people have both IFG and IGT (National Diabetes Statistics, 2007).

### SUMMARY OF THE CLAIMED INVENTION

In one aspect, the invention provides an isolated monoclonal antibody that binds to an epitope within human IAPP, such as an epitope within amino acid residues 1-10. The epitope can include an intact disulfide bridge between the cysteines at positions 2 and 7, with the disulfide bridge contributing to the epitope. The antibody can compete with monoclonal antibody 8A9 for binding to human IAPP and/or bind to the same epitope as monoclonal antibody 8A9. Subjects receiving such an antibody (e.g., subjects having or are at risk of a condition associated with IAPP accumulation) preferably have lower blood glucose levels following oral glucose challenge and/or reduced accumulation of IAPP relative to control subjects who did not receive the antibody or who received a control antibody. Preferably, the antibody is chimeric, veneered, humanized or human. The antibody can be of human IgG1 isotype. Alternatively, the antibody can be of human IgG2 or IgG4 isotype. The antibody can include at least one mutation (e.g., one or more mutations) in a constant region. The antibody can be an antigen-binding fragment, such as a Fab fragment or a single-chain Fv antibody.

In another aspect, the invention provides an antibody that includes three light chain CDRs (as defined by Kabat and/or Chothia) and three heavy chain CDRs (as defined by Kabat and/or Chothia) of monoclonal antibody 8A9. Subjects receiving such an antibody (e.g., subjects having or are at risk of a condition associated with IAPP accumulation) preferably have lower blood glucose levels following oral glucose challenge and/or reduced accumulation of IAPP relative to control subjects who did not receive the antibody or who received a control antibody. Preferably, the antibody is chimeric, veneered, or humanized. The antibody can be of human IgG1 isotype. Alternatively, the antibody can be of human IgG2 or IgG4 isotype. The antibody can include at least one mutation (e.g., one or more mutations) in a constant region. The antibody can be an antigen-binding fragment, such as a Fab fragment or a single-chain Fv antibody.

In another aspect, the invention provides an antibody comprising a mature heavy chain variable region having an amino acid sequence at least 90% identical (e.g., at least 95%, 98%, 99%, or 100% identical) to H7 (SEQ ID NO: 19) and a mature light chain variable region having an amino acid sequence at least 90% identical (e.g., at least 95%, 98%, 99%, or 100% identical) to L1 (SEQ ID NO: 35), wherein the antibody specifically binds to human IAPP. The antibody can include three CDRs of SEQ ID NO: 7 (e.g., as shown in SEQ ID NOs: 8-10) and three CDRs of SEQ ID NO: 29 (e.g., as shown in SEQ ID NOs: 30-32). In some antibodies, any differences in CDRs of the mature heavy chain variable region and mature light chain variable region from H7 and L1 (SEQ ID NOS: 19 and 35, respectively) reside in positions H60-H65. In some antibodies, H1 in the heavy chain variable region (Kabat numbering) is occupied by E. In some antibodies, at least one of positions H16, H17, H37, H46, H48, H69, H71, H73, H74, H75, H78, H79, H82C, and H85 in the heavy chain variable region (Kabat numbering) is occupied by Q, S, V, E, L, I, K, N, S, Q, V, F, L, and D, respectively, and/or at least one of positions L36, L44, L46, L66, L69, L71, and L85 in the light chain variable region (Kabat numbering) is occupied by L, I, R, R, S, Y, and D, respectively. In some antibodies, positions H16, H17, H46, H74, and H82C are occupied by Q, S, E, S, and L, respectively. In some antibodies, positions H37, H48, H69, H71, H73, H75, H78, and H79 are occupied by V, L, I, K, N, Q, V, and F, respectively. In some antibodies, positions H37, H69, H71, H78, H79, and H85 are occupied by V, I, K, V, F, and D, respectively. In some antibodies, positions H16, H17, H37, H46, H48, H69, H71, H73, H74, H75, H78, H79, and H82C in the heavy chain variable region (Kabat numbering) are occupied by Q, S, V, E, L, I, K, N, S, Q, V, F, and L, respectively. In some antibodies, positions H1, H16, H17, H37, H46, H48, H69, H71, H73, H74, H75, H78, H79, and H82C in the heavy chain variable region (Kabat numbering) are occupied by E, Q, S, V, E, L, I, K, N, S, Q, V, F, and L, respectively. In some antibodies, positions H16, H17, H37, H46, H69, H71, H74, H78, H79, H82C, and H85 in the heavy chain variable region (Kabat numbering) are occupied by Q, S, V, E, I, K, S, V, F, L, and D, respectively. In some antibodies, positions L36, L44, L46, L66, L69, and L71 in the light chain variable region (Kabat numbering) are occupied by L, I, R, R, S, and Y, respectively. In some antibodies, positions L36, L44, L46, L66, L69, L71, and L85 in the light chain variable region (Kabat numbering) are occupied by L, I, R, R, S, Y, and D, respectively. In some antibodies, positions H16, H17, H37, H46, H48, H69, H71, H73, H74, H75, H78, H79, and H82C in the heavy chain variable region (Kabat numbering) are occupied by Q, S, V, E, L, I, K, N, S, Q, V, F, and L, respectively, and positions L36, L44, L46, L66, L69, and L71 in the light chain variable region (Kabat numbering) are occupied by L, I, R, R, S, and Y, respectively. In some antibodies, positions H16, H17, H37, H46, H48, H69, H71, H73, H74, H75, H78, H79, and H82C in the heavy chain variable region (Kabat numbering) are occupied by Q, S, V, E, L, I, K, N, S, Q, V, F, and L, respectively, and positions L36, L44, L46, L66, L69, L71, and L85 in the light chain variable region (Kabat numbering) are occupied by L, I, R, R, S, Y, and D, respectively.

In some antibodies, the mature heavy chain variable region has an amino acid sequence designated H7 (SEQ ID NO: 19) and the mature light chain variable region has an amino acid sequence designated L1 (SEQ ID NO: 35). In other antibodies, the mature heavy chain variable region has an amino acid sequence designated H7 (SEQ ID NO: 19) and the mature light chain variable region has an amino acid sequence designated L3 (SEQ ID NO: 37)

In some antibodies, the mature heavy chain variable region is fused to a heavy chain constant region and/or the mature light chain variable region is fused to a light chain constant region. The heavy chain constant region can be a mutant form of a natural human heavy chain constant region which has reduced binding to an Fcγ receptor relative to the natural human heavy chain constant region. The heavy chain constant region can be of IgG1 isotype. In some antibodies, the mature heavy chain variable region is fused to a heavy chain constant region having the sequence of SEQ ID NO: 42 and/or the mature light chain variable region is fused to a light chain constant region having the sequence of SEQ ID NO: 44. In some antibodies, the mature heavy chain variable region is fused to a heavy chain constant region having the sequence of SEQ ID NO: 42, 46, or 47 and/or the mature light chain variable region is fused to a light chain constant region having the sequence of SEQ ID NO: 44 or 49. Alternatively, the antibody can be an antigen-binding fragment, such as a Fab fragment.

Any antibody of the invention can be provided in pure form (e.g., at least 95% w/w pure).

In another aspect, the invention provides pharmaceutical compositions that include an antibody of the invention and a pharmaceutically-acceptable carrier.

In another aspect, the invention provides a nucleic acid encoding the heavy and/or light chain(s) of an antibody of the invention, such as an antibody comprising a mature heavy chain variable region having an amino acid sequence at least 90% identical (e.g., at least 95%, 98%, 99%, or 100% identical) to H7 (SEQ ID NO: 19) and a mature light chain variable region having an amino acid sequence at least 90% identical (e.g., at least 95%, 98%, 99%, or 100% identical) to L1 (SEQ ID NO: 35), wherein the antibody specifically binds to human IAPP. For example, the heavy chain can be encoded by the nucleic acid sequence of SEQ ID NO: 27 and/or the light chain can be encoded by the nucleic acid sequence of SEQ ID NO: 38 or 40. Alternatively, the heavy chain can be encoded by the nucleic acid sequence of SEQ ID NO: 28 and/or the light chain can be encoded by the nucleic acid sequence of SEQ ID NO: 38 or 40.

In another aspect, the invention provides a recombinant expression vector comprising a nucleic acid encoding the heavy and/or light chain(s) of an antibody of the invention.

In another aspect, the invention provides a host cell transformed with a nucleic acid of the invention and/or a recombinant expression vector of the invention.

In another aspect, the invention provides a method of humanizing an antibody, particularly a mouse 8A9 antibody such as disclosed herein. The method includes determining the sequences of the heavy and light chain variable regions of a mouse antibody; synthesizing a nucleic acid encoding a humanized heavy chain comprising CDRs of the mouse antibody heavy chain and a nucleic acid encoding a humanized light chain comprising CDRs of the mouse antibody light chain; and expressing the nucleic acids in a host cell to produce a humanized antibody.

In another aspect, the invention provides a method of producing a humanized, chimeric, or veneered form of the mouse 8A9 antibody disclosed herein. The method includes culturing cells transformed with nucleic acids encoding the heavy and light chains of the humanized, chimeric, or veneered antibody, so that the cell secretes the antibody; and purifying the antibody from cell culture media.

In another aspect, the invention provides a method of producing a cell line that produces a humanized, chimeric, or veneered form of the mouse 8A9 antibody disclosed herein. The method includes introducing a vector encoding heavy and light chains of a humanized, chimeric, or veneered 8A9 antibody and a selectable marker into cells; propagating the cells under conditions to select for cells having increased copy number of the vector; isolating single cells from the selected cells; and banking cells cloned from a single cell selected based on yield of antibody.

In another aspect, the invention provides a method of making an antibody. The method includes obtaining a host cell of the invention; and maintaining the host cell under conditions in which the antibody is expressed. The method can further include collecting the antibody.

In another aspect, the invention provides a method of testing one or more antibodies, particularly an anti-IAPP antibody of the invention, as potential therapeutics. For each anti-IAPP test antibody, the method includes administering the test antibody to one or more transgenic rodents producing human IAPP (“IAPP transgenic rodents”); performing an oral glucose tolerance test on the one or more IAPP transgenic rodents; and comparing blood glucose levels in the IAPP transgenic rodents receiving the test antibody to blood glucose levels in control IAPP transgenic rodents that did not receive any antibody or that received a control antibody; and selecting the test antibody for development as a potential therapeutic if the blood glucose levels in IAPP transgenic rodents receiving the test antibody are significantly lower than the blood glucose levels in the control IAPP transgenic rodents. The antibody can be an 8A9 antibody. The development can include humanization of the test antibody. The IAPP transgenic rodent can be a HIP rat.

In some methods, 10 mg/kg of test antibody is administered to each rodent weekly. The test antibody can be administered for a period of at least 5, 10, 15, 16, 17, 18, 19, 20, 21, 22, 23, 24, 25, 26, 27, 28, 29, or 30 weeks. In some methods, control rodents are administered a control antibody according to the same schedule as the test antibody is administered to the rodents. Preferably, the control antibody has the same isotype as the test antibody.

In some methods, blood glucose levels are determined 120 minutes after glucose administration during the oral glucose tolerance test. In some methods, blood glucose levels are determined 30, 60, 90, 120, and/or 180 minutes after glucose administration during the oral glucose tolerance test.

In another aspect, the invention provides a method of reducing islet amyloid polypeptide (IAPP) accumulation in a subject having or at risk of a condition associated with IAPP accumulation. The method includes administering to the subject an effective regime of an antibody of the invention (e.g., a humanized or chimeric 8A9 antibody), thereby reducing IAPP accumulation in the subject.

In another aspect, the invention provides a method of inhibiting aggregation of islet amyloid polypeptide (IAPP) in a subject having or at risk of a condition associated with IAPP accumulation. The method includes administering to the subject an effective regime of an antibody of the invention (e.g., a humanized or chimeric 8A9 antibody), thereby inhibiting aggregation of IAPP in the subject.

In another aspect, the invention provides a method of stabilizing a non-toxic conformation of islet amyloid polypeptide (IAPP) in a subject having or at risk of a condition associated with IAPP accumulation. The method includes administering to the subject an effective regime of an antibody of the invention (e.g., a humanized or chimeric 8A9 antibody), thereby stabilizing a non-toxic conformation of IAPP in the subject.

In another aspect, the invention provides a method of reducing islet amyloid polypeptide (IAPP) deposits in a subject having or at risk of developing IAPP deposits. The method includes administering to the subject an effective regime of an antibody of the invention (e.g., a humanized or chimeric 8A9 antibody), thereby reducing IAPP deposits in the subject.

In another aspect, the invention provides a method of clearing aggregated islet amyloid polypeptide in a subject having or at risk of a condition associated with IAPP accumulation. The method includes administering to the subject an effective regime of an antibody of the invention (e.g., a humanized or chimeric 8A9 antibody), thereby clearing aggregated IAPP from the subject.

In another aspect, the invention provides a method of reducing glucose levels in a subject having Type 2 Diabetes (T2D). The method includes administering to the subject an effective regime of an antibody of the invention (e.g., a humanized or chimeric 8A9 antibody), thereby reducing glucose levels in the subject relative to a subject having T2D who has not received the antibody.

In another aspect, the invention provides a method of stabilizing glucose levels in a subject having Type 2 Diabetes (T2D). The method includes administering to the subject an effective regime of an antibody of the invention (e.g., a humanized or chimeric 8A9 antibody), thereby stabilizing glucose levels in the subject. In some methods, the glucose levels are fasting glucose levels. In some methods, the glucose levels are in response to an oral glucose challenge.

In another aspect, the invention provides a method of treating or effecting prophylaxis of a condition associated with IAPP amyloid accumulation in a subject. The method includes administering to the subject an effective regime of an antibody of the invention (e.g., a humanized or chimeric 8A9 antibody). In some methods the condition is associated with amyloid accumulation in the pancreas of the subject. In some methods the condition is type 2 diabetes. In some methods the condition is insulinoma. In some methods the condition is reduced Beta cell levels.

In another aspect, the invention provides a method of stabilizing Beta cell numbers, or delaying a decrease or reducing the rate of decrease in Beta cell numbers, in a subject having or at risk of a condition associated with IAPP accumulation. The method includes administering to the subject an effective regime of an antibody of the invention (e.g., a humanized or chimeric 8A9 antibody). In some methods, the number of Beta cells is determined from one or more sections of the pancreas, such as from a pancreatic biopsy.

In another aspect, the invention provides a method of increasing Beta cell numbers in a subject having or at risk of a condition associated with IAPP accumulation. The method includes administering to the subject an effective regime of an antibody of the invention (e.g., a humanized or chimeric 8A9 antibody). In some methods, the number of Beta cells is determined from one or more sections of the pancreas, such as from a pancreatic biopsy.

In another aspect, the invention provides a method for reducing inflammation associated with IAPP amyloid accumulation in a subject. The method includes administering to the subject an effective amount of an antibody of the invention (e.g., a humanized or chimeric 8A9 antibody). In some methods, the amyloid accumulation is in the pancreas of the subject.

In another aspect, the invention provides a method of reducing, ameliorating or preventing impaired glucose tolerance in a subject having or at risk of a condition associated with IAPP accumulation. The method includes administering to the subject an effective regime of an antibody of the invention (e.g., a humanized or chimeric 8A9 antibody).

In another aspect, the invention provides a method of diagnosing the presence or absence of an IAPP amyloid accumulation in a pancreas of a subject. The method includes contacting a sample from the subject suspected of comprising the amyloid accumulation with an effective amount of an antibody of the invention (e.g., a 8A9 antibody). Some methods also include detecting the binding of antibody to IAPP.

In another aspect, the invention provides a method of determining a level of IAPP deposits in a subject. The method include administering an antibody of the invention (e.g., a humanized or chimeric 8A9 antibody); and detecting the presence of bound antibody in the subject. In some methods, the presence of bound antibody is determined by positron emission tomography (PET).

In another aspect, the invention provides a method for delaying the onset of a condition associated with amyloid accumulation in a subject. The method includes administering to the subject an effective amount of a pharmaceutical composition described herein (e.g., a pharmaceutical composition comprising a humanized 8A9 antibody). In some methods, the condition is associated with amyloid accumulation in the pancreas of the subject. In some methods, the condition is type 2 diabetes. In some methods, the condition is insulinoma. In some methods, the condition is reduced Beta cell numbers.

In another aspect, the invention provides a method of reducing beta islet cellular toxicity associated with aggregates or oligomers of IAPP. The method includes administering an effective regime of a pharmaceutical composition described herein (e.g., a pharmaceutical composition comprising a humanized 8A9 antibody).

In another aspect, the invention provides a method of delaying the progression in a subject from pre-diabetes to diabetes. The method includes administering an effective regime of a pharmaceutical composition described herein (e.g., a pharmaceutical composition comprising a humanized 8A9 antibody).

In another aspect, the invention provides a method of ameliorating impaired fasting glucose (IFG) in a subject. The method includes administering an effective regime of a pharmaceutical composition described herein (e.g., a pharmaceutical composition comprising a humanized 8A9 antibody).

In another aspect, the invention provides a method of ameliorating impaired glucose tolerance (IGT) in a subject. The method includes administering an effective regime of a pharmaceutical composition described herein (e.g., a pharmaceutical composition comprising a humanized 8A9 antibody).

In another aspect, the invention provides a method of stabilizing fasting blood glucose levels in a subject at less than 100 milligrams per deciliter after an overnight fast. The method includes administering an effective regime of a pharmaceutical composition described herein (e.g., a pharmaceutical composition comprising a humanized 8A9 antibody).

In another aspect, the invention provides a method of reducing glucose levels in a subject having Type 1 Diabetes (T1D). The method includes administering to the subject an effective regime of an antibody of the invention (e.g., a humanized or chimeric 8A9 antibody), thereby reducing glucose levels in the subject relative to a subject having T1D who has not received the antibody.

In another aspect, the invention provides a method of stabilizing glucose levels in a subject having Type 1 Diabetes (T1D). The method includes administering to the subject an effective regime of an antibody of the invention (e.g., a humanized or chimeric 8A9 antibody), thereby stabilizing glucose levels in the subject. In some methods, the glucose levels are fasting glucose levels. In some methods, the glucose levels are in response to an oral glucose challenge.

In another aspect, the invention provides a method of reducing glucose levels in a subject having Type 1.5 Diabetes (T1.5D). The method includes administering to the subject an effective regime of an antibody of the invention (e.g., a humanized or chimeric 8A9 antibody), thereby reducing glucose levels in the subject relative to a subject having T1.5D who has not received the antibody.

In another aspect, the invention provides a method of stabilizing glucose levels in a subject having Type 1.5 Diabetes (T1.5D). The method includes administering to the subject an effective regime of an antibody of the invention (e.g., a humanized or chimeric 8A9 antibody), thereby stabilizing glucose levels in the subject. In some methods, the glucose levels are fasting glucose levels. In some methods, the glucose levels are in response to an oral glucose challenge.

In any of the above methods of treating a subject or detecting IAPP in a subject, the subject can be a human.

### BRIEF DESCRIPTION OF THE DRAWINGS

FIG. 1 depicts hemoglobin A1c levels in the blood of the HIP rats after twenty-two weeks of treatment with either 8A9 antibody or PS2 isotype control antibody.

FIGS. 2A and 2B depict blood glucose levels in HIP rats after twenty-four weeks of treatment with either 8A9 antibody or PS2 isotype control antibody. The blood glucose levels were measured as part of an oral glucose challenge in which 2 g/kg of glucose was ingested after an overnight fast. The individual panels show the blood glucose levels measured at 90 minutes (FIG. 2A) and 180 minutes (FIG. 2B) after administration of glucose. FIG. 2C is a graph of the results of the glucose challenge test. The graph shows the average blood glucose levels just prior to the oral glucose challenge (time 0) and 30, 60, 90, 120, and 180 minutes after ingestion of glucose. The error bars in FIGS. 2A-C represent one standard deviation from the mean.

FIGS. 3A, 3B, and 3C depict blood glucose levels in HIP rats after twenty-eight weeks of treatment with either 8A9 antibody or PS2 isotype control antibody. The blood glucose levels were measured as part of an oral glucose challenge in which 2 g/kg of glucose was ingested after an overnight fast. The individual panels show the blood glucose levels measured at 30 minutes (FIG. 3A), 60 minutes (FIG. 3B), and 180 minutes (FIG. 3C) after administration of glucose. FIG. 3D is a graph of the results of the glucose challenge test. The graph shows the average blood glucose levels just prior to the oral glucose challenge (time 0) and 30, 60, 90, 120, and 180 minutes after ingestion of glucose. The error bars in FIGS. 3A-D represent one standard deviation from the mean.

FIG. 4 depicts the average percentage of the pancreas having IAPP amyloid deposits in HIP rats after thirty weeks of treatment with either 8A9 antibody or PS2 isotype control antibody.

FIG. 5 depicts the average percentage of the pancreas having Beta cells in HIP rats after thirty weeks of treatment with either 8A9 antibody or PS2 isotype control antibody.

### BRIEF DESCRIPTION OF THE SEQUENCES

SEQ ID NO:1 is the mature human IAPP (hIAPP) sequence.

SEQ ID NO: 2 is a peptide corresponding to amino acid residues 1-10 of hIAPP.

SEQ ID NO: 3 is a nucleic acid sequence encoding the 8A9 heavy chain variable region.

SEQ ID NO: 4 is the 8A9 heavy chain variable region sequence, including signal peptide.

SEQ ID NO: 5 is a nucleic acid sequence encoding the 8A9 light chain variable region.

SEQ ID NO: 6 is the 8A9 light chain variable region sequence, including signal peptide.

SEQ ID NO: 7 is the 8A9 mature heavy chain variable region sequence.

SEQ ID NO: 8 is the 8A9 heavy chain CDR1, according to composite Kabat/Chothia numbering.

SEQ ID NO: 9 is the 8A9 heavy chain CDR2, according to Kabat numbering.

SEQ ID NO: 10 is the 8A9 heavy chain CDR3, according to Kabat numbering.

SEQ ID NO: 11 is the murine VH Sequence from the PTB 2GJZ structural model (for antibody 13G5).

SEQ ID NO: 12 is the human VH Acceptor framework of Acc#BAC02410.

SEQ ID NO: 13 is the humanized 8A9 H1 sequence.

SEQ ID NO: 14 is the humanized 8A9 H2 sequence.

SEQ ID NO: 15 is the humanized 8A9 H3 sequence.

SEQ ID NO: 16 is the humanized 8A9 H4 sequence.

SEQ ID NO: 17 is the humanized 8A9 H5 sequence.

SEQ ID NO: 18 is the humanized 8A9 H6 sequence.

SEQ ID NO: 19 is the humanized 8A9 H7 sequence.

SEQ ID NO: 20 is the humanized 8A9 H7b sequence.

SEQ ID NO: 21 is a nucleic acid sequence encoding the humanized 8A9 H1 region.

SEQ ID NO: 22 is a nucleic acid sequence encoding the humanized 8A9 H2 region

SEQ ID NO: 23 is a nucleic acid sequence encoding the humanized 8A9 H3 region.

SEQ ID NO: 24 is a nucleic acid sequence encoding the humanized 8A9 H4 region.

SEQ ID NO: 25 is a nucleic acid sequence encoding the humanized 8A9 H5 region.

SEQ ID NO: 26 is a nucleic acid sequence encoding the humanized 8A9 H6 region.

SEQ ID NO: 27 is a nucleic acid sequence encoding the humanized 8A9 H7 region.

SEQ ID NO: 28 is a nucleic acid sequence encoding the humanized 8A9 H7b region.

SEQ ID NO: 29 is the 8A9 mature light chain variable region sequence.

SEQ ID NO: 30 is the 8A9 light chain CDR1, according to Kabat numbering.

SEQ ID NO: 31 is the 8A9 light chain CDR2, according to Kabat numbering.

SEQ ID NO: 32 is the 8A9 light chain CDR3, according to Kabat numbering.

SEQ ID NO: 33 is the murine VL Sequence from the PTB 2RCS structural model (for antibody 28B4).

SEQ ID NO: 34 is the human VL acceptor framework of Acc#AAZ09096.1.

SEQ ID NO: 35 is the humanized 8A9 L1 sequence.

SEQ ID NO: 36 is the humanized 8A9 L2 sequence.

SEQ ID NO: 37 is the humanized 8A9 L3 sequence.

SEQ ID NO: 38 is a nucleic acid sequence encoding the humanized 8A9 L1 region.

SEQ ID NO: 39 is a nucleic acid sequence encoding the humanized 8A9 L2 region.

SEQ ID NO: 40 is a nucleic acid sequence encoding the humanized 8A9 L3 region.

SEQ ID NO: 41 is a nucleic acid sequence encoding an exemplary human IgG1 constant region.

SEQ ID NO: 42 is an exemplary human IgG1 constant region.

SEQ ID NO: 43 is a nucleic acid sequence encoding an exemplary human kappa light chain constant region without an N-terminal arginine.

SEQ ID NO: 44 is an exemplary human kappa light chain constant region without an N-terminal arginine.

SEQ ID NO: 45 is a nucleic acid sequence encoding an exemplary human IgG1 constant region of the G1m3 allotype.

SEQ ID NO: 46 is an exemplary human IgG1 constant region of the G1m3 allotype.

SEQ ID NO: 47 is an exemplary human IgG1 constant region of the G1m3 allotype.

SEQ ID NO: 48 is a nucleic acid sequence encoding an exemplary human kappa light chain constant region with an N-terminal arginine.

SEQ ID NO: 49 is an exemplary human kappa light chain constant region with an N-terminal arginine.

### DEFINITIONS

The term “antibody” includes intact antibodies and binding fragments thereof. Typically, fragments compete with the intact antibody from which they were derived for specific binding to the target. Fragments include separate heavy chains, separate light chains, Fab, Fab′, F(ab′)2, F(ab)c, Fv, single chain antibodies, and single domain antibodies. Single (variable) domain antibodies include VH regions separated from their VL partners (or vice versa) in conventional antibodies (Ward et al., 1989, Nature 341: 544-546), as well as VH regions (sometimes known as VHH) from species such as Camelidae or cartilaginous fish (e.g., a nurse shark) in which VH regions are not associated with VL regions (see, e.g., WO 9404678). Single domain antibodies in which one chain is separated from its natural partners are sometimes known as Dabs and single domain antibodies from Camelidae or cartilaginous fish are sometimes known as nanobodies. Constant regions or parts of constant regions may or may not be present in single domain antibodies. For example, natural single variable region antibodies from Camelidae include a VHH variable region, and CH2 and CH3 constant regions. Single domain antibodies, such as nanobodies, can be subject to humanization by analogous approaches to conventional antibodies. Dabs antibodies are usually obtained from antibodies of human origin. Fragments can be produced by recombinant DNA techniques, or by enzymatic or chemical separation of intact immunoglobulins. The term “antibody” also includes a bispecific antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol., 148:1547-53 (1992)).

An “antigen” is an entity to which an antibody specifically binds.

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, X-ray crystallography and 2-dimensional nuclear magnetic resonance. See, e.g., Epitope Mapping Protocols, in Methods in Molecular Biology, Vol. 66, Glenn E. Morris, Ed. (1996). An epitope can include a C-terminal residue or an N-terminal residue. An epitope can also include, but need not include, the free amino group of a polypeptide or the free carboxyl group of a polypeptide. Thus, an epitope can include a C-terminal or an N-terminal residue, but not necessarily include the free carboxyl group or the free amino group, respectively. Antibody binding specificity is sometimes defined by a range of amino acids. If an antibody is said to bind to an epitope within amino acids 1-10 of SEQ ID NO:1, for example, what is meant is that the epitope is within the recited range of amino acids including those defining the outer-limits of the range. It does not necessarily mean that every amino acid within the range constitutes part of the epitope. Thus, for example, an epitope within amino acids 1-10 of SEQ ID NO:1 may consist of amino acids 1-8, 2-9, 2-10, or 2-7, among other segments of SEQ ID NO:1.

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen. See, e.g., Junghans et al. (1990), Cancer Res. 50:1495. A test antibody competes with a reference antibody if an excess of a test antibody (e.g., at least 2×, 5×, 10×, 20× or 100×) inhibits binding of the reference antibody by at least 50%, 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “subject” includes a human and other mammal (e.g., non-human primate, canine, feline, mouse, rat, bovine, equine, and porcine) that receives either prophylactic or therapeutic treatment with an agent such as an antibody or an immunogen.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions (with gaps not counted) multiplied by 100 to convert to percentage.

For purposes of classifying amino acids substitutions as conservative or non-conservative, amino acids are grouped as follows: Group I (hydrophobic side chains): Norleucine, Met, Ala, Val, Leu, Ile; Group II (neutral hydrophilic side chains): Cys, Ser, Thr; Group III (acidic side chains): Asp, Glu; Group IV (basic side chains): Asn, Gln, His, Lys, Arg; Group V (residues influencing chain orientation): Gly, Pro; and Group VI (aromatic side chains): Trp, Tyr, Phe. Conservative substitutions involve substitutions between amino acids in the same class. Non-conservative substitutions constitute exchanging a member of one of these classes for a member of another.

Antibodies of the invention typically bind to their designated target with an affinity constant of at least 10⁶, 10⁷, 10⁸, 10⁹, or 10¹⁰ M. Such binding is specific binding in that it is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a monoclonal antibody binds one and only one target.

The term “symptom” refers to subjective evidence of a disease, such as altered gait, as perceived by a subject. A “sign” refers to objective evidence of a disease as observed by a physician.

An individual is at increased risk of a disease if the subject has at least one known risk-factor (e.g., genetic, biochemical, family history, situational exposure) placing individuals with that risk factor at a statistically significant greater risk of developing the disease than individuals without the risk factor. Statistical significance means p<0.05.

Monoclonal antibodies and other therapeutic agents (e.g., immunogens) are typically provided in isolated form. This means that the agent is typically at least 50% w/w pure of interfering proteins and other contaminants arising from its production or purification, but does not exclude the possibility that the agent is combined with an excess of pharmaceutically-acceptable carrier(s) or other vehicle intended to facilitate its use. Sometimes monoclonal antibodies are at least 60%, 70%, 80%, 90%, 95%, 96%, 97%, 98%, or 99% w/w pure of interfering proteins and contaminants from production or purification.

The basic antibody structural unit is a tetramer of subunits. Each tetramer includes two identical pairs of polypeptide chains, each pair having one “light” chain (about 25 kDa) and one “heavy” chain (about 50-70 kDa). The amino-terminal portion of each chain includes a variable region of about 100 to 110 or more amino acids primarily responsible for antigen recognition. When initially expressed, this variable region is typically linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function. A constant region can include any or all of a CH1 region, hinge region, CH2 region, and CH3 region.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites. Except for bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the regions FR1, CDR1, FR2, CDR2, FR3, CDR3 and FR4. The assignment of amino acids to each region is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chains or between different light chains are assigned the same number.

A disease, condition, or disorder “associated with IAPP accumulation” or “associated with IAPP amyloid accumulation” refers to a disease, condition, or disorder for which at least one symptom is associated with an abnormal accumulation of a deposit that includes a statistically significant level of IAPP.

“Metabolic syndrome” is a term of art used to describe a disorder comprising combinations of type 2 diabetes, glucose tolerance, impaired insulin sensitivity, hypertension, obesity, increased abdominal girth, hypertriglyceridemia, low HDL, hyperuricaemia, hypercoagulability and/or microalbuminemia. The International Diabetes Federation consensus worldwide definition of the metabolic syndrome (2006) is: Central obesity AND any two of the following: raised triglycerides: >150 mg/dL (1.7 mmol/L), or specific treatment for this lipid abnormality; reduced HDL cholesterol (<40 mg/dL (1.03 mmol/L) in males and <50 mg/dL (1.29 mmol/L) in females), or specific treatment for this lipid abnormality; raised blood pressure (systolic BP>130 or diastolic BP>85 mm Hg), or treatment of previously diagnosed hypertension; raised fasting plasma glucose (FPG) (>100 mg/dL (5.6 mmol/L)); or previously diagnosed type 2 diabetes.

“Impaired insulin sensitivity” is a disorder in which one or more of the body's normal physiological responses to insulin are impaired or lost. Impaired insulin sensitivity in a subject is characterized by a reduced biological response to endogenous or exogenous insulin. Impaired insulin sensitivity is associated with a number of diseases or disorders in humans, including increased risk of developing type 2 diabetes. Impaired insulin sensitivity is also a feature of metabolic syndrome, which is a cluster of abnormalities that create risk for many of our most common medial diseases or disorders. Impaired insulin sensitivity can be determined by methods such as the oral glucose tolerance test (OGTT), IV glucose tolerance test (FSIVGTT), insulin tolerance test (ITT), insulin sensitivity test (1ST), and continuous infusion of glucose with model assessment (CIGMA), or the glucose clamp. See, e.g., Krentz, Insulin Resistance (Wiley-Blackwell, 2002); de Paula Martins et al., Eur. J. Obst. Gynecol. Reprod. Biol., 133 (2):203-207. Obesity, Body Mass Index (BMI) and Visceral Adiposity.

“Diabetes” is a disorder generally characterized by metabolic defects in production and utilization of glucose that result in the failure to maintain appropriate blood sugar levels in the body. The result of these defects is elevated blood glucose, referred to as “hyperglycemia.” Diabetes in humans can be defined as a disorder corresponding to a fasting plasma glucose concentration greater than 125 mg/dl, or a plasma glucose concentration greater than 199 mg/dl two hours after ingestion of a 75 g oral glucose load. Two major forms of diabetes are type 1 diabetes and type 2 diabetes. Type 1 diabetes is generally the result of an absolute deficiency of insulin, the hormone that regulates glucose utilization. Type 2 diabetes often occurs in the face of normal, or even elevated levels of insulin and can result from the inability of tissues to respond appropriately to insulin. Most Type 2 diabetic patients are insulin resistant (i.e., having impaired insulin sensitivity) and have a relative deficiency of insulin, in that insulin secretion cannot compensate for the resistance of peripheral tissues to respond to insulin. In addition, many type 2 diabetics are obese. Type 1.5 diabetes (late autoimmune onset in adults) shows some characteristics of type 1 and type 2 diabetes.

“Glucose tolerance” refers to a state of proper functioning of the homeostatic mechanisms by which insulin is secreted in response to an elevation in blood glucose concentrations. Impairment in this system results in transient hyperglycemia as the organism is unable to maintain normoglycemia following a glucose load (for example, a carbohydrate containing meal) because of insufficient secretion of insulin from the islet beta-cells or because of insensitivity of target tissues to circulating insulin. “Impaired glucose tolerance” in humans can be defined as a plasma glucose concentration greater than or equal to 140 mg/dl (7.8 mmol/1) two hours after ingestion of a 75 g oral glucose load.

“Beta cells” refers to pancreatic cells found in the islets of Langerhans that store and secrete insulin. A “Beta cell number” is a normalized measure of the number of Beta cells in the pancreas. A subject's Beta cell number can be determined, for example, by staining pancreatic tissue (e.g., sectioned tissue, optionally from a biopsy) for insulin (e.g., using anti-insulin antibodies).

### DETAILED DESCRIPTION

### I. General

The invention provides monoclonal antibody 8A9 and related antibodies. The 8A9 antibody binds to an epitope within residues 1-10 of IAPP, with an intact disulfide bridge between cysteines at positions 2 and 7 contributing to the epitope. The antibodies of the invention are useful, for example, for treating disorders associated with IAPP accumulation, particularly accumulation of IAPP deposits. Such disorders include type 2 diabetes, metabolic syndrome, impaired insulin tolerance, impaired glucose tolerance, insulinomas, and related conditions.

### II. IAPP

Unless otherwise apparent from the context, reference to IAPP (or hIAPP) means human islet-amyloid polypeptide, which is a 37 amino acid peptide having the sequence: KCNTATCATQRLANFLVHSSNNFGAILSSTNVGSNTY (SEQ ID NO: 1).

IAPP is originally synthesized as residues 34-70 of an 89-amino acid precursor protein (Swiss Prot P10997) of which residues 1-22 are a signal peptide, and residues 23-31 and 74-89 are propeptides. Mature IAPP can form “oligomers”, which are soluble multimeric assemblies of two or more IAPP peptides.

### III. Antibodies

8A9 is an exemplary antibody of the invention, whose heavy and light chain mature variable regions are designated SEQ ID NO: 7 and SEQ ID NO: 29, respectively. The invention also provides antibodies competing with 8A9 for binding to IAPP, or which bind to the same or overlapping epitope as an antibody designated 8A9 (i.e., within residues 1-10 of IAPP and with an intact disulfide bridge between residues 2 and 7) and have similar functional properties, such as stabilizing blood glucose levels and thereby reducing, ameliorating, or preventing impaired glucose tolerance.

Other antibodies having such a binding specificity can be produced by immunizing mice with IAPP or a fragment thereof (i.e., a fragment including amino acid residues 1-10 with an intact disulfide bridge, or a portion thereof including the intact disulfide bridge), and screening the resulting antibodies for binding to IAPP, optionally in competition with 8A9. Antibodies can also be screened for their effect (1) in IAPP transgenic rodent models subjected to oral glucose challenge or other test, (2) on rodent or other non-human animal model for a disease characterized by IAPP accumulation by oral glucose challenge or other test, and/or (3) in humans with a condition associated with IAPP accumulation by oral glucose challenge or other test. Alternatively, or in addition to any of the foregoing approaches, antibodies can be screened against mutagenized forms of IAPP to identify an antibody showing the same or similar binding profile as 8A9 to a collection of mutational changes. The mutations can be systematic substitution with alanine (or serine if an alanine is present already) one residue at a time, or more broadly spaced intervals, throughout IAPP or through a section thereof in which the epitope is known to reside (i.e., residues 1-10).

Antibodies having the binding specificity of a selected murine antibody (e.g., 8A9) can also be produced using a variant of the phage display method. See Winter, WO 92/20791. This method is particularly suitable for producing human antibodies. In this method, either the heavy or light chain variable region of the selected murine antibody is used as a starting material. If, for example, a light chain variable region is selected as the starting material, a phage library is constructed in which members display the same light chain variable region (i.e., the murine starting material) and a different heavy chain variable region. The heavy chain variable regions can for example be obtained from a library of rearranged human heavy chain variable regions. A phage showing strong specific binding for IAPP (e.g., at least 10⁸M⁻¹, and preferably at least 10⁹ M⁻¹) is selected. The heavy chain variable region from this phage then serves as a starting material for constructing a further phage library. In this library, each phage displays the same heavy chain variable region (i.e., the region identified from the first display library) and a different light chain variable region. The light chain variable regions can be obtained for example from a library of rearranged human variable light chain regions. Again, phage showing strong specific binding for IAPP are selected. The resulting antibodies usually have the same or similar epitope specificity as the murine starting material.

Other antibodies can be obtained by mutagenesis of cDNA encoding the heavy and light chains of an exemplary antibody, such as 8A9. Accordingly, monoclonal antibodies that are at least 70%, 80%, 90%, 95%, 96%, 97%, 98% or 99% identical to 8A9 in amino acid sequence of the mature heavy and/or light chain variable regions and maintain its functional properties, and/or which differ from the respective antibody by a small number of functionally inconsequential amino acid substitutions (e.g., conservative substitutions), deletions, or insertions are also included in the invention.

The invention also includes monoclonal antibodies having at least 3, 4, 5, and preferably all six CDR(s) as defined by Kabat that are 90%, 95%, 99% or 100% identical to corresponding CDRs of 8A9. The invention also includes monoclonal antibodies having at least 3, 4, 5, and preferably all six CDR(s) as defined by Chothia that are 90%, 95%, 99% or 100% identical to corresponding CDRs of 8A9. The invention also includes monoclonal antibodies having at least 3, 4, 5 and preferably all six CDR(s) as shown in Tables 9 and 10 that are 90%, 95%, 99% or 100% identical to corresponding CDRs of 8A9. Such monoclonal antibodies preferably have at least 70%, 80%, 90%, 95%, 96%, 97%, 98% or 99% identity to 8A9 in amino acid sequence of the mature heavy and/or light chain variable regions and maintain its functional properties, and/or differ from 8A9 by a small number of functionally inconsequential amino acid substitutions (e.g., conservative substitutions), deletions, or insertions.

Preferred antibodies show similar functional activity to 8A9, e.g., in stabilizing blood glucose and/or hemoglobin A1c levels, reducing IAPP deposits or accumulation, and/or stabilizing Beta cell numbers in a human with or at risk of any condition associated with IAPP accumulation disclosed herein or in an animal model thereof. A level of blood glucose or hemoglobin A1c is considered “stabilized” if, when measured under specific conditions, the level in a subject with a condition associated with IAPP accumulation does not increase (beyond experimental error) over time or, increases at a lower rate than in an untreated control subject over the same period of time. Thus, stabilization of blood glucose levels can be shown when, under conditions such as fasting or following an oral glucose challenge, one or more measurements of blood glucose in an afflicted subject (e.g., human subject) treated with an anti-IAPP antibody of the invention (e.g., for a period of at least 18 weeks) is not higher (beyond experimental error) than corresponding prior measurement(s) of blood glucose in the afflicted subject (e.g., prior to treatment), or at least one or more measurements of blood glucose in the afflicted subject is lower than corresponding measurement(s) of blood glucose in an afflicted control subject (e.g., a subject that has received, over the same period of time, a control antibody, a placebo, or no treatment at all). Similarly, stabilization of hemoglobin A1c levels can be shown when a measurement of hemoglobin A1c in an afflicted subject (e.g., human subject) treated with an anti-IAPP antibody of the invention (e.g., for a period of at least 18 weeks) is not greater (beyond experimental error) than a corresponding prior measurement of hemoglobin A1c in the afflicted subject (e.g., prior to treatment), or the measurement of hemoglobin A1c in the afflicted subject is lower than a corresponding measurement of hemoglobin A1c in an afflicted control subject (e.g., a subject that has received, over the same period of time, a control antibody, a placebo, or no treatment at all). A reduction in IAPP deposits or accumulation means that the percentage of the pancreas having IAPP deposits in an afflicted subject (e.g., human subject) treated with an antibody of the invention (e.g., for a period of at least 18 weeks) is lower (e.g., by a statistically significant amount) than in an afflicted control subject (e.g., a subject that has received, over the same period of time, a control antibody, a placebo, or no treatment at all). A stabilization of Beta cell numbers means that the Beta cell number in an afflicted subject (e.g., human subject) treated with an antibody of the invention (e.g., for a period of at least 18 weeks) does not decrease (beyond experimental error) over a period of time (e.g., the period of treatment).

Preferably, treatment with an anti-IAPP antibody of the invention is for a sufficient period of time (e.g., for 5, 10, 15, 18, 20, 22, 24, 26, 28, 30, or more weeks) such that, after a 120 minute oral glucose challenge, the afflicted subject's blood glucose level is the same as (within experimental error) or less than a prior corresponding measurement of blood glucose (e.g., an oral glucose challenge measurement taken earlier in the treatment or prior to the start of treatment) in the same subject. Preferably, treatment with an anti-IAPP antibody of the invention (e.g., for 5, 10, 15, 18, 20, 22, 24, 26, 28, 30, or more weeks) stabilizes the blood glucose levels of an afflicted subject such that, at one or more time points after an oral glucose challenge (e.g., 30′, 60′, 90′, 120′, 150′, and/or 180′), the afflicted subject's blood glucose levels are the same as (within experimental error) or less than prior corresponding measurements of blood glucose in the same subject.

Preferably, treatment with an anti-IAPP antibody of the invention is for a sufficient period of time (e.g., for 5, 10, 15, 18, 20, 22, 24, 26, 28, 30, or more weeks) such that, after a 120 minute oral glucose challenge, the afflicted subject's blood glucose level is less than a corresponding measurement of blood glucose in an afflicted control subject. Preferably, treatment with an anti-IAPP antibody of the invention (e.g., for 5, 10, 15, 18, 20, 22, 24, 26, 28, 30, or more weeks) stabilizes the blood glucose levels of an afflicted subject such that, at one or more time points after an oral glucose challenge (e.g., 30′, 60′, 90′, 120′, 150′, and/or 180′), the afflicted subject's blood glucose levels are less than corresponding measurements of blood glucose in an afflicted control subject.

Preferably, treatment with an anti-IAPP antibody of the invention (e.g., for 5, 10, 15, 18, 20, 22, 24, 26, 28, 30, or more weeks) stabilizes the hemoglobin A1c level of an afflicted subject such that the afflicted subject's hemoglobin A1c level is the same as (within experimental error) or less than a prior measurement of hemoglobin A1c in the same subject (e.g., a measurement taken earlier in the treatment or prior to the start of treatment). Preferably, treatment with an anti-IAPP antibody of the invention (e.g., for 5, 10, 15, 18, 20, 22, 24, 26, 28, 30, or more weeks) stabilizes the hemoglobin A1c level of an afflicted subject such that the afflicted subject's hemoglobin A1c level is less than the corresponding measurement of blood glucose in an afflicted control subject.

Stabilization preferably occurs while a subject is receiving a recurring treatment regime and continues for at least 3, 6, or 12 months, or indefinitely.

Preferably, treatment with an anti-IAPP antibody of the invention (e.g., for 5, 10, 15, 18, 20, 22, 24, 26, 28, 30, or more weeks) reduces IAPP accumulation (or deposits) in an afflicted subject such that the amount of IAPP accumulation in the afflicted subject is the same as (within experimental error) or less than a prior measurement of IAPP accumulation in the same subject (e.g., a measurement taken earlier in the treatment or prior to the start of treatment). Preferably, treatment with an anti-IAPP antibody of the invention (e.g., for 5, 10, 15, 18, 20, 22, 24, 26, 28, 30, or more weeks) reduces IAPP accumulation in an afflicted subject such that the afflicted subject's level of IAPP accumulation is less than a corresponding measure of IAPP accumulation in an afflicted control subject.

Preferably, treatment with an anti-IAPP antibody of the invention (e.g., for 5, 10, 15, 18, 20, 22, 24, 26, 28, 30, or more weeks) stabilizes or increases the Beta cell number in an afflicted subject such that the Beta cell number in the afflicted subject is the same as (within experimental error) or higher than a prior measurement of Beta cell number in the same subject (e.g., a measurement taken earlier in the treatment or prior to the start of treatment). Preferably, treatment with an anti-IAPP antibody of the invention (e.g., for 5, 10, 15, 18, 20, 22, 24, 26, 28, 30, or more weeks) stabilizes a Beta cell number in an afflicted subject such that the afflicted subject's Beta cell number is greater than a corresponding measure of Beta cell number in an afflicted control subject.

Exemplary methods of measuring stabilization of blood glucose levels, reduction in hemoglobin A1c, and reduction in IAPP accumulation in the pancreas in the rat HIP model are provided in the Examples that follow. Blood glucose levels and hemoglobin A1c levels can be measured, e.g., in whole blood, serum, or plasma. IAPP accumulation can be measured, e.g., in pancreas sections stained with thioflavine T. Regardless of the method, the difference between blood glucose levels, hemoglobin A1c levels, or IAPP accumulation in an afflicted treated subject and an afflicted control subject, under the specified conditions, should be statistically significant or otherwise beyond experimental error.

A. Chimeric and Veneered Antibodies

The invention further provides chimeric and veneered forms of non-human antibodies, particularly 8A9.

A chimeric antibody is an antibody in which the mature variable regions of light and heavy chains of a non-human antibody (e.g., a mouse) are combined with light and heavy chain constant regions from an antibody of a different species. Typically, the light and heavy chain constant regions are of human origin, but the constant regions can originate from a different non-human species, such as a rat, as needed (e.g., to facilitate testing of the non-human antibody in an appropriate animal model). Such antibodies substantially or entirely retain the binding specificity of the non-human (e.g., mouse) antibody supplying the variable regions, and are about two-thirds human (or different non-human species) sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions. Veneered forms of 8A9 are included in the invention.

B. Humanized Antibodies

A humanized antibody is a genetically engineered antibody in which the CDRs from a non-human “donor” antibody (e.g., 8A9) are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. No. 5,859,205 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly, a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. And a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and Dabs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85%, 90%, 95%, or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs) from a mouse antibody. See, e.g., Pascalis et al. (20020, J. Immunol. 169:3076; Vajdos et al. (2002), Journal of Molecular Biology, 320: 415-428; Iwahashi et al. (1999), Mol. Immunol. 36:1079-1091; and Tamura et al. (2000), Journal of Immunology, 164:1432-1441.

In some antibodies only part of the CDRs, namely the subset of CDR residues required for binding, termed the SDRs, are needed to retain binding in a humanized antibody. CDR residues not contacting antigen and not in the SDRs can be identified based on previous studies (for example residues H60-H65 in CDR H2 are often not required), from regions of Kabat CDRs lying outside Chothia hypervariable loops (Chothia, J. Mol. Biol. 196:901, 1987), by molecular modeling and/or empirically, or as described in Gonzales et al. (2004), Mol. Immunol. 41: 863. For such humanized antibodies, at positions in which one or more donor CDR residues are absent or in which an entire donor CDR is omitted, the amino acid occupying the position can be an amino acid occupying the corresponding position (by Kabat numbering) in the acceptor antibody sequence. The number of substitutions of acceptor for donor amino acids in the CDRs that can be included reflects a balance of competing considerations. Such substitutions are potentially advantageous in decreasing the number of mouse amino acids in a humanized antibody and consequently decreasing potential immunogenicity. However, substitutions can also cause changes of affinity, and significant reductions in affinity are preferably avoided. Positions for substitution within CDRs and amino acids to substitute can also be selected empirically.

The human acceptor antibody sequences can optionally be selected from among the many known human antibody sequences to provide a high degree of sequence identity (e.g., 65%-85% identity) between the human acceptor sequence variable region frameworks and corresponding variable region frameworks of the donor antibody chain.

Certain amino acids from the human variable region framework residues can be selected for substitution (i.e., backmutation) based on their possible influence on CDR conformation and/or binding to antigen. Investigation of such possible influences is by modeling, examination of the characteristics of the amino acids at particular locations, or empirical observation of the effects of substitution or mutagenesis of particular amino acids.

For example, when an amino acid differs between a murine variable region framework residue and a selected human variable region framework residue, the human framework amino acid can be substituted by the equivalent framework amino acid from the mouse antibody when it is reasonably expected that the amino acid:

(1) noncovalently binds antigen directly,

(2) is adjacent to a CDR region,

(3) otherwise interacts with a CDR region (e.g. is within about 6 Å of a CDR region), (e.g., identified by modeling the light or heavy chain on the solved structure of a homologous known immunoglobulin chain); and

(4) a residue participating in the VL-VH interface.

Framework residues from classes (1)-(3) as defined by Queen (U.S. Pat. No. 5,530,101) can be alternately referred to as canonical or vernier residues. Framework residues that help determine the conformation of a CDR loop are sometimes referred to as canonical residues (Chothia and Lesk, J. Mol. Biol. 196, 901-917 (1987), Thornton & Martin J. Mol. Biol., 263, 800-815, 1996). Framework residues that support antigen-binding loop conformations and play a role in fine-tuning the fit of an antibody to antigen are sometimes referred to as vernier residues (Foote & Winter, 1992, J Mol Bio. 224, 487-499).

Other framework residues that are candidates for substitution are residues creating a potential glycosylation site. Still other candidates for substitution are acceptor human framework amino acids that are unusual for a human immunoglobulin at that position. These amino acids can be substituted with amino acids from the equivalent position of the mouse donor antibody or from the equivalent positions of more typical human immunoglobulins.

The invention provides humanized forms of the mouse 8A9 antibody. The mouse antibody comprises mature heavy and light chain variable regions having amino acid sequences comprising SEQ ID NO: 7 and SEQ ID NO: 29, respectively. The invention provides eight exemplified humanized mature heavy chain variable regions (H1, SEQ ID NO: 13; H2, SEQ ID NO: 14; H3, SEQ ID NO: 15; H4, SEQ ID NO: 16; H5, SEQ ID NO: 17; H6, SEQ ID NO: 18; H7, SEQ ID NO: 19; and H7b, SEQ ID NO: 20) and three exemplified humanized mature light chain variable region (L1, SEQ ID NO: 35; L2, SEQ ID NO: 36; and L3, SEQ ID NO: 37). The H7L1 variant, which includes nineteen backmutations, provides an affinity to IAPP that is about 2.5 nM, which is within a factor of 1.5 of the affinity of the mouse 8A9 antibody, which is within the margin of error in the assay. The H7L3 variant, which includes twenty backmutations, provides an affinity to IAPP that is about 3.7 nM, which is within a factor of 2 of the affinity of the mouse 8A9 antibody, which is also within the margin of error in the assay.

The invention provides variants of the H7L1 humanized 8A9 antibody in which the humanized mature heavy chain variable region shows at least 90%, 95%, 96%, 97%, 98%, or 99% identity to H7 (SEQ ID NO: 19) and the humanized mature light chain variable region shows at least 90%, 95%, 96%, 97%, 98%, or 99% sequence identity to L1 (SEQ ID NO: 35). In some such antibodies, one, two, three, four, five, six, seven, eight, nine, ten, eleven, twelve, thirteen, fourteen, fifteen, sixteen, seventeen, eighteen, or nineteen of the backmutations in H7L1 are retained. In some antibodies, position H1 in the Vh region is occupied by a E. In some antibodies, position H16 in the Vh region is occupied by a Q, position H17 in the Vh region is occupied by a S, position H37 in the Vh region is occupied by a V, position H46 in the Vh region is occupied by a E, position H48 in the Vh region is occupied by a L, position H69 in the Vh region is occupied by a I, position H71 in the Vh region is occupied by a K, position H73 in the Vh region is occupied by a N, position H74 in the Vh region is occupied by a S, position H75 in the Vh region is occupied by a Q, position H78 in the Vh region is occupied by a V, position H79 in the Vh region is occupied by a F, position H82C in the Vh region is occupied by a L, and/or position H85 in the Vh region is occupied by a D. In some antibodies, position L36 in the Vk region is occupied by L, position L44 in the Vk region is occupied by I, position L46 in the Vk region is occupied by R, position L66 in the Vk region is occupied by R, position L69 in the Vk region is occupied by S, position L71 in the Vk region is occupied by Y, and/or position L85 in the Vk region is occupied by D. Some antibodies have position H16 occupied by a Q, position H17 occupied by a S, position H46 occupied by a E, position H74 occupied by a S, and position H82C occupied by a L. Some antibodies have position H37 occupied by a V, position H48 occupied by a L, position H69 occupied by a I, position H71 occupied by a K, position H73 occupied by a N, position H75 occupied by a Q, position H78 occupied by a V, and position H79 occupied by a F. Some antibodies have position H37 occupied by a V, position H69 occupied by a I, position H71 occupied by a K, position H78 occupied by a V, position H79 occupied by a F, and position H85 occupied by a D. Some antibodies have position L36 occupied by a L, position L44 occupied by a I, position L46 occupied by a R, position L66 occupied by a R, position L69 occupied by a S, and position L71 occupied by a Y. Some antibodies have position L36 occupied by a L, position L44 occupied by a I, position L46 occupied by a R, position L66 occupied by a R, position L69 occupied by a S, position L71 occupied by a Y, and position L85 occupied by a D. Some antibodies have position H16 occupied by a Q, position H17 occupied by a S, position H37 occupied by a V, position H46 occupied by a E, position H48 occupied by a L, position H69 occupied by a I, position H71 occupied by a K, position H73 occupied by a N, position H74 occupied by a S, position H75 occupied by a Q, position H78 occupied by a V, position H79 occupied by a F, and position H82C occupied by a L, some of which additional have position L36 occupied by a L, position L44 occupied by a I, position L46 occupied by a R, position L66 occupied by a R, position L69 occupied by a S, and position L71 occupied by a Y. Some antibodies have position H1 occupied by a E, H16 occupied by a Q, position H17 occupied by a S, position H37 occupied by a V, position H46 occupied by a E, position H48 occupied by a L, position H69 occupied by a I, position H71 occupied by a K, position H73 occupied by a N, position H74 occupied by a S, position H75 occupied by a Q, position H78 occupied by a V, position H79 occupied by a F, and position H82C occupied by a L, some of which additional have position L36 occupied by a L, position L44 occupied by a I, position L46 occupied by a R, position L66 occupied by a R, position L69 occupied by a S, and position L71 occupied by a Y. The CDR regions of such humanized antibodies can be identical or substantially identical to the CDR regions of H7L1, which are the same as those of the mouse donor antibody. The CDR regions can be defined by any conventional definition (e.g., Kabat or Chothia) but are preferably as defined by the shaded regions shown in Tables 9 and 10.

One possibility for additional variation in humanized 8A9 variants is additional backmutations in the variable region frameworks. Many of the framework residues not in contact with the CDRs in the humanized mAb can accommodate substitutions of amino acids from the corresponding positions of the donor mouse mAb or other mouse or human antibodies, and even many potential CDR-contact residues are also amenable to substitution or even amino acids within the CDRs may be altered, for example, with residues found at the corresponding position of the human acceptor sequence used to supply variable region frameworks. In addition, alternate human acceptor sequences can be used, for example, for the heavy and/or light chain. If different acceptor sequences are used, one or more of the backmutations recommended above may not be performed because the corresponding donor and acceptor residues are already the same without backmutation.

The invention also includes humanized antibodies in which the mature light and heavy chain variable regions shows at least 90, 95, 96, 97, 98 or 99% sequence identity to the mature light and heavy chain variable regions of the humanized 8A9 HILL H1L2, H1L3, H2L1, H2L2, H2L3, H3L1, H3L2, H3L3, H4L1, H4L2, H4L3, H5L1, H5L2, H5L3, H6L1, H6L2, H6L3, H7L2, H7L3, H7bL1, H7bL2, or H7bL3.

C. Selection of Constant Region

The heavy and light chain variable regions of chimeric, humanized (including veneered), or human antibodies can be linked to at least a portion of a constant region sufficient to interact with an Fc receptor. The constant region is typically human, but a non-human (e.g., rat) constant region can be selected as needed.

The choice of constant region depends, in part, on whether antibody-dependent complement and/or cellular mediated cytotoxicity is desired. For example, human isotopes IgG1 and IgG3 have complement-mediated cytotoxicity whereas human isotypes IgG2 and IgG4 have poor or no complement-mediated cytotoxicity. A human IgG1 constant region suitable for inclusion in the antibodies of the invention can have the sequence of SEQ ID NO: 42. Light chain constant regions can be lambda or kappa. A human kappa light chain constant region suitable for inclusion in the antibodies of the invention can have the sequence of SEQ ID NO: 44. Another human kappa light chain constant region suitable for inclusion in the antibodies of the invention can have the sequence of SEQ ID NO: 49, which differs from SEQ ID NO: 44 in that it has the addition of an N-terminal arginine. Antibodies can be expressed as tetramers containing two light and two heavy chains, as separate heavy chains, as separate light chains, as Fab, Fab′, F(ab′)₂, or Fv fragments, or as single chain antibodies in which heavy and light chain variable regions are linked through a spacer.

Human constant regions show allotypic variation and isoallotypic variation between different individuals. That is, the constant regions can differ in different individuals at one or more polymorphic positions. Isoallotypes differ from allotypes in that serum recognizing an isoallotype binds to a non-polymorphic region of one or more other isotypes. Thus, for example, another heavy chain constant region is of the IgG1 G1m3 allotype and has the amino acid sequence of SEQ ID NO: 46. Another heavy chain constant region of the IgG1 G1m3 allotype has the amino acid sequence of SEQ ID NO: 47. Reference to a human constant region includes a constant region with any natural allotype or any permutation of residues occupying polymorphic positions in natural allotypes or up to 3, 5 or 10 substitutions for reducing or increasing effector function as described below.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering is used in this paragraph for the constant region) for increasing the half-life of an antibody. Substitution at any or all of positions 234, 235, 236 and/or 237 reduces affinity for Fcy receptors, particularly FcyRI receptor (see, e.g., U.S. Pat. No. 6,624,821). An alanine substitution at positions 234, 235 and 237 of human IgG1 can be used for reducing effector functions. Optionally, positions 234, 236 and/or 237 in human IgG2 are substituted with alanine and position 235 with glutamine. (See, e.g., U.S. Pat. No. 5,624,821.). In some aspects, a mutation at one or more of positions 241, 264, 265, 270, 296, 297, 322, 329, and 331 by EU numbering of human IgG1 is used. In some aspects, a mutation at one or more of 318, 320, and 322 by EU numbering of human IgG1 is used. In some aspects, positions 234 and/or 235 are substituted with alanine and/or position 329 is substituted with glycine. In some aspects, positions 234 and 235 are substituted with alanine, such as in SEQ ID NO: 47. In some aspects, the isotype is human IgG2 or IgG4.

D. Human Antibodies

Human antibodies against IAPP are provided by a variety of techniques described below. Some human antibodies are selected by competitive binding experiments, or otherwise, to have the same or overlapping epitope specificity as 8A9. Human antibodies can also be screened for a particular epitope specificity by using only a fragment of IAPP (e.g., residues 3-12) as the immunogen, and/or by screening antibodies against a collection of deletion mutants of IAPP. One technique for producing human antibodies is trioma methodology (Oestberg et al., Hybridoma 2:361-367 (1983); Oestberg, U.S. Pat. No. 4,634,664; and Engleman et al., U.S. Pat. No. 4,634,666). Another technique involves immunizing transgenic mice expressing human immunoglobulin genes, such as the XenoMouse®, AlivaMab Mouse or Veloceimmune mouse (see, e.g., Lonberg et al., W093/1222, U.S. Pat. No. 5,877,397, U.S. Pat. No. 5,874,299, U.S. Pat. No. 5,814,318, U.S. Pat. No. 5,789,650, U.S. Pat. No. 5,770,429, U.S. Pat. No. 5,661,016, U.S. Pat. No. 5,633,425, U.S. Pat. No. 5,625,126, U.S. Pat. No. 5,569,825, U.S. Pat. No. 5,545,806, Nature 148, 1547-1553 (1994), Nature Biotechnology 14, 826 (1996), Kucherlapati, WO 91/10741. Another technique is phage display (see, e.g., Dower et al., WO 91/17271 and McCafferty et al., WO 92/01047, U.S. Pat. No. 5,877,218, U.S. Pat. No. 5,871,907, U.S. Pat. No. 5,858,657, U.S. Pat. No. 5,837,242, U.S. Pat. No. 5,733,743 and U.S. Pat. No. 5,565,332. In these methods, libraries of phage are produced in which members display different antibodies on their outer surfaces. Antibodies are usually displayed as Fv or Fab fragments. Phage displaying antibodies with a desired specificity are selected by affinity enrichment to an IAPP peptide or fragment thereof. Another technique is to sequence DNA from human B cells according to the general protocols outlined in Reddy et al., Nat Biotechnol. 2010 September; 28(9):965-9. Epub 2010 August 29; and US 20110053803, 20100099103, 20100291066, 20100035763, and 20100151471. Briefly, B cells can be obtained from a human suspected of having anti-IAPP antibodies, e.g., a human immunized with IAPP, fragments thereof, longer polypeptides containing IAPP or fragments thereof, or anti-idiotypic antibodies. The mRNA of the antibodies from B cells is then reverse transcribed into cDNA and sequenced using, e.g., 454 sequencing technology. After obtaining the sequences of the chains from each antibody, the chains can be paired together (e.g., using bioinformatics), cloned, expressed, and screened for desired properties.

E. Expression of Recombinant Antibodies

A number of methods are known for producing chimeric and humanized antibodies using an antibody-expressing cell line (e.g., hybridoma). For example, the immunoglobulin variable regions of antibodies can be cloned and sequenced using well known methods. In one method, the heavy chain variable VH region is cloned by RT-PCR using mRNA prepared from hybridoma cells. Consensus primers are employed to VH region leader peptide encompassing the translation initiation codon as the 5′ primer and a g2b constant regions specific 3′ primer. Exemplary primers are described in U.S. patent publication US 2005/0009150 by Schenk et al. (hereinafter, “Schenk”). The sequences from multiple, independently-derived clones, can be compared to ensure no changes are introduced during amplification. The sequence of the VH region can also be determined or confirmed by sequencing a VH fragment obtained by 5′ RACE RT-PCR methodology and the 3′ g2b specific primer.

The light chain variable VL region can be cloned in an analogous manner as the VH region. In one approach, a consensus primer set designed for amplification of VL regions is designed to hybridize to the VL region encompassing the translation initiation codon, and a 3′ primer specific for the Ck region downstream of the V-J joining region. In a second approach, 5′RACE RT-PCR methodology is employed to clone a VL encoding cDNA. Exemplary primers are described in Schenk, supra. The cloned sequences are then combined with sequences encoding human (or other non-human species) constant regions. Exemplary sequences encoding human constant regions include SEQ ID NO: 41, which encodes a human IgG1 constant region, and SEQ ID NO: 43, which encodes a human kappa light chain constant region. Other exemplary sequences encoding human constant regions include SEQ ID NO: 45, which encodes a human IgG1 constant region of the G1m3 allotype, and SEQ ID NO: 48, which encodes a human kappa light chain constant region.

In one approach, the heavy and light chain variable regions are re-engineered to encode splice donor sequences downstream of the respective VDJ or VJ junctions, and cloned into the mammalian expression vector, such as pCMV-hγ1 for the heavy chain, and pCMV-Mcl for the light chain. These vectors encode human γ1 and Ck constant regions as exonic fragments downstream of the inserted variable region cassette. Following sequence verification, the heavy chain and light chain expression vectors can be co-transfected into CHO cells to produce chimeric antibodies. Conditioned media is collected 48 hrs. post-transfection and assayed by western blot analysis for antibody production or ELISA for antigen binding. The chimeric antibodies are humanized as described above.

Chimeric, veneered, humanized, and human antibodies are typically produced by recombinant expression. Recombinant polynucleotide constructs typically include an expression control sequence operably linked to the coding sequences of antibody chains, including naturally associated or heterologous expression control element(s), such as a promoter. Once the vector has been incorporated into the appropriate host, the host is maintained under conditions suitable for high level expression of the nucleotide sequences, and the collection and purification of the cross reacting antibodies.

These expression vectors are typically replicable in the host organisms either as episomes or as an integral part of the host chromosomal DNA. Commonly, expression vectors contain selection markers, e.g., ampicillin-resistance or hygromycin-resistance, to permit detection of those cells transformed with the desired DNA sequences.

E. coli is one prokaryotic host useful for cloning the DNA sequences encoding the polypeptides disclosed herein. Microbes, such as yeast are also useful for expression. Saccharomyces is a yeast host, with suitable vectors having expression control sequences, an origin of replication, termination sequences and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells are a host cell for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed, and include CHO cell lines, various COS cell lines, HeLa cells, L cells, human embryonic kidney cell, and myeloma cell lines. The cells can be nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can include promoters derived from endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. No. 5,741,957, U.S. Pat. No. 5,304,489, U.S. Pat. No. 5,849,992). Suitable transgenes include coding sequences for light and/or heavy chains in operable linkage with a promoter and enhancer from a mammary gland specific gene, such as casein or beta lactoglobulin.

The vectors containing the DNA segments of interest can be transferred into the host cell by methods depending on the type of cellular host. For example, calcium chloride transfection is commonly utilized for prokaryotic cells, whereas calcium phosphate treatment, electroporation, lipofection, biolistics or viral-based transfection can be used for other cellular hosts. Other methods used to transform mammalian cells include the use of polybrene, protoplast fusion, liposomes, electroporation, and microinjection. For production of transgenic animals, transgenes can be microinjected into fertilized oocytes, or can be incorporated into the genome of embryonic stem cells, and the nuclei of such cells transferred into enucleated oocytes.

Having introduced vector(s) encoding antibody heavy and light chains into cell culture, cell pools can be screened for growth productivity and product quality in serum-free media. Top-producing cell pools can then be subjected of FACS-based single-cell cloning to generate monoclonal lines. Specific productivities above 50 pg or 100 pg per cell per day, which correspond to product titers of greater than 7.5 g/L culture, can be used. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or Biacore. A selected clone can then be banked in multiple vials and stored frozen for subsequent use.

Once expressed, antibodies can be purified according to standard procedures of the art, including protein A capture, HPLC purification, column chromatography, gel electrophoresis and the like (see generally, Scopes, Protein Purification (Springer-Verlag, NY, 1982)).

Methodology for commercial production of antibodies can be employed, including codon optimization, selection of promoters, transcription elements, and terminators, serum-free single cell cloning, cell banking, use of selection markers for amplification of copy number, CHO terminator, serum free single cell cloning, improvement of protein titers (see, e.g., U.S. Pat. No. 5,786,464, U.S. Pat. No. 6,114,148, U.S. Pat. No. 6,063,598, U.S. Pat. No. 7,569,339, WO2004/050884, WO2008/012142, WO2008/012142, WO2005/019442, WO2008/107388, and WO2009/027471, and U.S. Pat. No. 5,888,809).

F. Antibody Screening Assays

Antibodies can be subject to several screens including binding assays, functional screens, screens in animal models of diseases associated with IAPP deposits, and clinical trials. Binding assays test for specific binding and, optionally, affinity and epitope specificity to IAPP (or a fragment thereof, such as amino acid residues 1-10). Such screens are sometimes performed in competition with an exemplary antibody, such as 8A9. Optionally, either the antibody or IAPP target is immobilized in such assay. Functional assays can be performed in cellular models including cells naturally expressing IAPP or transfected with DNA encoding IAPP or a fragment thereof. Suitable cells include cells derived from pancreatic islet cells. Cells can be screened for reduced levels of IAPP (e.g., by Western blotting or immunoprecipitation of cell extracts or supernatants) or reduced toxicity attributable to IAPP.

Animal model screens test the ability of the antibody to therapeutically or prophylactically treat signs or symptoms in an animal model simulating a human disease associated with IAPP deposits, such as type II diabetes or impaired glucose tolerance. Suitable signs or symptoms that can be monitored include elevated blood glucose levels (e.g., fasting blood glucose levels or blood glucose levels following an oral glucose challenge) and/or elevated hemoglobin A1c levels and/or reduced levels of IAPP amyloid or accumulation (e.g., in the pancreas). The extent of elevation can be determined by comparison with an appropriate control, such as blood glucose levels in control animals that have received a control antibody (e.g., an isotype matched control antibody), a placebo, or no treatment at all. Transgenic or other animal models of type 2 diabetes include HIP rats, db/db mouse, Zucker diabetic fatty rat, ob/ob mouse, high calorie-fed Psammomys obesus (sand rat), Goto-Katazaki rat (GK rat), and RIPHAT transgenic mice. Transgenic animals can include a human IAPP transgene. To facilitate testing in animal models, chimeric antibodies having a constant region appropriate for the animal model can be used (e.g., mouse-rat chimeras could be used for testing antibodies in HIP rats). It can be concluded that a humanized version of an antibody will be effective if the corresponding mouse antibody or chimeric antibody is effective in an appropriate animal model and the humanized antibody has similar binding affinity (e.g., within experimental error, such as by a factor of 1.5, 2, or 3).

Clinical trials test for safety and efficacy in a human having a disease associated with IAPP deposits.

### IV. Pharmaceutical Compositions and Methods of Use

Provided herein are several methods of diagnosing, monitoring, treating or effecting prophylaxis of diseases or conditions associated with IAPP deposition (e.g., IAPP accumulation) or toxic IAPP oligomers. Examples of such diseases include Type 2 diabetes and related conditions, including metabolic syndrome, impaired insulin sensitivity, impaired glucose tolerance, or insulinomas. Antibodies described above can be incorporated into pharmaceutical composition for use in such methods. In general, an antibody or pharmaceutical composition containing an antibody is administered to a subject in need thereof. Patients amenable to treatment include individuals at risk of an IAPP associated disease but not showing symptoms, as well as patients presently showing symptoms. Therefore, the pharmaceutical compositions can be administered prophylactically to individuals who have a known genetic risk of an IAPP-associated disease. Such individuals include those having relatives who have experienced such a disease, and those whose risk is determined by analysis of genetic or biochemical markers, including the diagnostic methods provided herein. See, e.g., Janssens et al. (2006), Predictive genetic testing for type-2 diabetes, BMJ, 333:509-510; Saxena et al., (2010), Nat Gen, 42:142-148. Besides family history and genetics, low activity level, poor diet, and excess body weight (especially around the waist) significantly increase the risk of developing type 2 diabetes. Other risk factors include: an age greater than 45 years; an HDL cholesterol of less than 35 mg/dL or triglyceride level of greater than 250 mg/dL; high blood pressure; history of gestational diabetes; previously identified impaired glucose tolerance; and race/ethnicity (African Americans, Hispanic Americans, and Native Americans all have high rates of diabetes). Though often no symptoms are shown, individuals suffering from T2D can sometimes be recognized from its clinical manifestations including high blood glucose levels, blurred vision, erectile dysfunction, fatigue, frequent or slow-healing infections, increased appetite, increased thirst, increased urination. As warranted by family history, genetic testing or medical screening for type-2 diabetes, treatment can begin at any age (e.g., 10, 20, 30). Usually, however, it is not necessary to begin treatment until a patient reaches 40, 50, 60 or 70. Treatment typically entails multiple dosages over a period of time and can be monitored by assaying antibody or activated T-cell or B-cell responses to a therapeutic agent (e.g., a truncated form of IAPP) over time. If the response falls, a booster dosage is indicated.

The identification of the subject can occur in a clinical setting, or elsewhere, e.g., in the subject's home, e.g., through the subject's own use of a self-testing kit. For example, the subject can be identified based on various symptoms such as increased thirst, increased frequency of urination, increased hunger, weight loss, fatigue, blurred vision, slow-healing sores, frequent infections, and areas of darkened skin. In some examples, the subject can be identified using a fasting blood glucose level test (e.g., testing to see if glucose is 100-125 mg/dL after an overnight or eight hour fast) or an oral glucose tolerance test (e.g., testing to see if glucose levels are 140-199 mg/dL two hours after taking a dose of a high-sugar solution).

In humans, fasting levels less than 100 mg/dL or oral glucose tolerance test levels less than 140 mg/dL are considered normal, levels of 100-125 (fasting) or 140-199 (oral glucose tolerances test) are considered impaired (pre-diabetic) and levels of >125 (fasting) or >199 (oral glucose tolerance test) are considered not only impaired but diabetic. In prophylactic applications, an antibody or a pharmaceutical composition of the same is administered to a subject susceptible to, or otherwise at risk of a disease (e.g., a Type 2 diabetes disease) in a regime (dose, frequency and route of administration) effective to reduce the risk, lessen the severity, or delay the onset of at least one sign or symptom of the disease. In therapeutic applications, an antibody or immunogen to induce an antibody is administered to a subject suspected of, or already suffering from a disease (e.g., a Type 2 diabetes disease) in a regime (dose, frequency and route of administration) effective to ameliorate or at least inhibit further deterioration of at least one sign or symptom of the disease.

A regime is considered therapeutically or prophylactically effective if an individual treated subject achieves an outcome more favorable than the mean outcome in a control population of comparable subjects not treated by methods disclosed herein, or if a more favorable outcome is demonstrated for a regime in treated subjects versus control subjects in a controlled clinical trial (e.g., a phase II, phase II/III, or phase III trial) or an animal model at the p<0.05 or 0.01 or even 0.001 level.

An effective regime of an antibody can be used for, e.g., reducing islet amyloid polypeptide (IAPP) accumulation in a subject having or at risk of a condition associated with IAPP accumulation; inhibiting aggregation of islet amyloid polypeptide (IAPP) in a subject having or at risk of a condition associated with IAPP accumulation; inhibiting toxic effects of IAPP oligomers in a subject having or at risk of a condition associated with toxic IAPP oligomers or IAPP accumulation; stabilizing a non-toxic conformation of islet amyloid polypeptide (IAPP) in a subject having or at risk of a condition associated with toxic conformations of IAPP or IAPP accumulation; reducing or clearing islet amyloid polypeptide deposits (IAPP) in a subject having or at risk of developing IAPP deposits; clearing aggregated islet amyloid polypeptide in a subject having or at risk of a condition associated with IAPP accumulation; stabilizing or reducing glucose levels in a subject having Type 1 Diabetes (T1D); stabilizing or reducing glucose levels in a subject having Type 1.5 Diabetes (T1.5D); stabilizing or reducing glucose levels in a subject having Type 2 Diabetes (T2D); reducing beta islet cellular toxicity associated with aggregates or oligomers of IAPP in a subject having or at risk of a condition associated with toxic conformations of IAPP or IAPP accumulation; reducing, ameliorating, or preventing impaired glucose tolerance in a subject having or at risk of a condition associated with toxic conformations of IAPP or IAPP accumulation; ameliorating impaired fasting glucose in a subject having or at risk of a condition associated with toxic conformations of IAPP or IAPP accumulation; treating or effecting prophylaxis of a condition associated with amyloid accumulation in a subject (e.g., a condition (e.g., T2D, metabolic syndrome, glucose intolerance, insulinomas or inflammation) associated with amyloid accumulation in the pancreas of the subject); reducing inflammation in a subject associated with amyloid accumulation in the subject, e.g., accumulation in the subject's pancreas; diagnosing the presence or absence of an amyloid accumulation in a pancreas by contacting a sample suspected of comprising the amyloid accumulation with an effective amount of an agent that binds to an epitope within the N-terminal region of IAPP; determining a level of IAPP deposits in a subject by detecting the presence of bound antibody in the subject following administration of the agent; inducing an immune response comprising antibodies to IAPP in a subject; delaying the onset of a condition associated with amyloid accumulation in a subject; preventing or delaying progression of pre-diabetes to diabetes in a subject with impaired fasting glucose (for example, having a fasting blood glucose level of 100 to 125 milligrams per deciliter after an overnight fast), impaired glucose tolerance (for example, having a blood glucose level of 140 to 199 milligrams per deciliter after a 2-hour oral glucose tolerance test) or both IFG and IGT; methods of stabilizing fasting blood glucose levels in a subject at less than 100 milligrams per deciliter after an overnight fast.

Effective doses vary depending on many different factors, such as means of administration, target site, physiological state of the subject, and whether the subject is human or an animal, other medications administered, and whether treatment is prophylactic or therapeutic.

An exemplary dose range for antibodies can be from about 0.01 to 10 mg/kg, and more usually 0.1 to 3 mg/kg or 0.15-2 mg/kg or 0.15-1.5 mg/kg, of subject body weight. Antibody can be administered in such doses daily, on alternative days, weekly, fortnightly, monthly, quarterly, or according to any other schedule determined by empirical analysis. An exemplary treatment entails administration in multiple doses over a prolonged period, for example, of at least six months. Additional exemplary treatment regimes entail administration once per every two weeks or once a month or once every 3 to 6 months.

Antibodies can be administered via a peripheral route (i.e., one in which an administered or induced antibody crosses the blood brain barrier to reach an intended site in the brain. Routes of administration include topical, intravenous, oral, subcutaneous, intraarterial, intracranial, intrathecal, intraperitoneal, intranasal or intramuscular. Routes for administration of antibodies can be intravenous or subcutaneous. This type of injection is most typically performed in the arm or leg muscles. In some methods, agents are injected directly into a particular tissue where deposits have accumulated, for example intracranial injection.

Pharmaceutical compositions for parenteral administration can be sterile and substantially isotonic and manufactured under GMP conditions. Pharmaceutical compositions can be provided in unit dose form (i.e., the dose for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, e.g., in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

The regimes can be administered in combination with another agent effective in treatment or prophylaxis of the disease being treated.

After treatment, the subject's condition can be evaluated to determine the progress or efficacy of such treatment. Such methods preferably test for stabilization in blood glucose levels. Such levels can be measured after a subject has fasted (e.g., for 8-16 hours). Alternatively, or in addition, the test can evaluate the blood glucose levels of a subject at a specified period (e.g., 2 hrs.) after ingestion of a specified quantity of glucose, e.g., 75 g (referred to as an oral glucose challenge test). The subject's blood glucose level may be evaluated to determine improvement (i.e., lower glucose level) relative to the subject's glucose level under comparable circumstances prior to treatment (i.e., fasting glucose level or glucose level after oral glucose challenge test). The subject's blood glucose level can also be compared with control populations under comparable circumstances. The control populations can be similarly afflicted, untreated subjects or normal untreated subjects (among other control subjects). Improvement (decreased levels) relative to similarly afflicted, untreated subjects or levels approaching or reaching the levels in untreated normal subjects indicates a positive response to treatment. Methods of measuring blood glucose levels and related kits are well-known.

The extent to which glucose levels are being controlled can be determined indirectly by measuring glycated hemoglobin levels in the blood, for example, hemoglobin A1c or Hb A1c levels. Other indirect measures of the extent to which glucose levels are being controlled include measuring blood insulin levels. Efficacy can also be monitored by assessing changes in IAPP amyloid levels by a number of methods, including imaging techniques. Examples of suitable imaging techniques include PET scanning with radiolabeled IAPP or fragments thereof, IAPP antibodies or fragments thereof, Congo red based amyloid imaging agents such as, for example, PiB (US 20110008255, amyloid-imaging peptide p31 (Biodistribution of amyloid-imaging peptide, p31, correlates with amyloid quantitation based on Congo red tissue staining, Wall et al., Abstract No. 1573, 2011 ISNM Annual Meeting) and other PET labels.

A. Diagnostics and Monitoring Methods

Also provided are methods of detecting an immune response against IAPP in a patient suffering from or susceptible to diseases associated with IAPP deposition or toxic IAPP oligomers. The methods can be used to monitor a course of therapeutic and prophylactic treatment with the agents provided herein. For example, the methods can be used to monitor active immunization (e.g., antibody produced in response to administration of immunogen) and passive immunization (e.g., measuring level of administered antibody).

Also provided are methods of detecting IAPP amyloid in a subject, for example, by measuring IAPP amyloid in a sample from a subject or by in vivo imaging of IAPP in a subject. Such methods are useful to diagnose or confirm diagnosis of diseases associated with IAPP, or susceptibility thereto. The methods can also be used on asymptomatic subjects. The presence of abnormal deposits of IAPP indicates susceptibility to future symptomatic disease. The methods are also useful for monitoring disease progression and/or response to treatment in subjects who have been previously diagnosed with an IAPP-associated disease.

Fluid or tissue samples obtained from a subject having, suspected of having, or at risk of having an IAPP-associated disease can be contacted with the antibodies disclosed herein to assess the presence of IAPP amyloid. For example, levels of IAPP amyloid in such subjects may be compared to those present in healthy subjects. Alternatively, levels of IAPP amyloid in such subjects receiving treatment for the disease may be compared to those of subjects who have not been treated for an IAPP-associated disease. Some such tests involve a biopsy of tissue obtained from the pancreas of such subjects. ELISA assays may also be useful methods, for example, for assessing IAPP levels in fluid samples. Some such ELISA assays involve IAPP antibodies that preferentially bind oligomeric or aggregated forms of IAPP relative to monomeric forms of IAPP.

The in vivo imaging methods can work by administering a reagent, such as antibody that binds to IAPP in the subject, and then detecting the reagent after it has bound. Antibodies typically bind to an epitope of IAPP within the N-terminal region of IAPP. If desired, the clearing response can be avoided by using antibody fragments lacking a full length constant region, such as Fabs. In some methods, the same antibody can serve as both a treatment and diagnostic reagent.

Diagnostic reagents can be administered by intravenous injection into the body of the subject, or via other routes deemed reasonable. The dose of reagent should be within the same ranges as for treatment methods. Typically, the reagent is labeled, although in some methods, the primary reagent with affinity for IAPP is unlabeled and a secondary labeling agent is used to bind to the primary reagent. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labeled loci to corresponding base line values. The base line values can represent the mean levels in a population of undiseased individuals. Base line values can also represent previous levels determined in the same subject. For example, base line values can be determined in a subject before beginning treatment, and measured values thereafter compared with the base line values. A decrease in values relative to base line generally signals a positive response to treatment.

B. Passive Immunization

The antibody profile following passive immunization typically shows an immediate peak in antibody concentration followed by an exponential decay. Without a further dose, the decay approaches pretreatment levels within a period of days to months depending on the half-life of the antibody administered. For example the half-life of some human antibodies is of the order of 20 days.

In some methods, a baseline measurement of antibody to IAPP in the subject is made before administration, a second measurement is made soon thereafter to determine the peak antibody level, and one or more further measurements are made at intervals to monitor decay of antibody levels. When the level of antibody has declined to baseline or a predetermined percentage of the peak less baseline (e.g., 50%, 25% or 10%), administration of a further dose of antibody is administered. In some methods, peak or subsequent measured levels less background are compared with reference levels previously determined to constitute a beneficial prophylactic or therapeutic treatment regime in other subjects. If the measured antibody level is significantly less than a reference level (e.g., less than the mean minus one or, preferably, two standard deviations of the reference value in a population of subjects benefiting from treatment) administration of an additional dose of antibody is indicated.

### V. Kits

Also provided are kits including an IAPP-specific antibody and instructions for use. Such kits can be used for, e.g., performing the diagnostic methods described above. A kit can also include a label. Kits also typically contain labeling providing directions for use of the kit. The labeling may also include a chart or other correspondence regime correlating levels of measured label with levels of antibodies to IAPP. The term labeling generally refers to any written or recorded material that is attached to, or otherwise accompanies a kit at any time during its manufacture, transport, sale or use. For example, the term labeling encompasses advertising leaflets and brochures, packaging materials, instructions, audio or video cassettes, computer discs, as well as writing imprinted directly on kits.

Also provided are diagnostic kits for performing in vivo imaging. Such kits typically contain an antibody binding to an epitope of IAPP as described herein. The antibody can be labeled or a secondary labeling reagent is included in the kit. The kit can include instructions for performing an in vivo imaging assay.

### EXAMPLES

### Example 1

### Isolation of Murine 8A9

Five week old female A/J mice were immunized intraperitoneally on day 0 and 7 with 25 μg of IAPP conjugated to Keyhole Limpet Hemocyanin through a glutaraldehyde conjugation. On day 14 and 21, the mice received 10 μg of IAPP/KLH conjugate all emulsified in RIBI adjuvant. On day 28, they were bled and titered against IAPP peptide and the highest titer mouse was immunized 10 days later, with IAPP/KLH in PBS given ½ IP and ½ intravenously.

Three days later the spleen was removed, a cell suspension was generated from the spleen, and the spleen cells were fused to SP2/0 cells. Resultant hybridomas were screened by ELISA against IAPP. Positives were further epitope mapped and 8A9 was found to react with amino acid residues 1-10 of IAPP (SEQ ID NO: 2), with a strong preference to an intact disulfide bridge between the cysteine at position 2 and 7.

### Example 2

### Effects of Antibody 8A9 in HIP Rats Following 22-28 Weeks of Treatment

To test that ability of the 8A9 antibody to alleviate abnormal glucose metabolism, such as associated with type 2 diabetes, the rat HIP model was selected. To facilitate such testing, chimeric mouse-rat 8A9 antibodies were generated.

### Mouse-Rat 8A9 Chimeric Antibodies

Briefly, the PS/2 rat hybridoma was purchased from ATCC. As per information provided by ATCC, the PS/2 hybridoma expresses an IgG2b/kappa isotype rat antibody. mRNA was purified from PS/2 cells using QiagenOligotex Direct mRNA kit. Purified mRNA was then directly used for PCR amplification of the constant regions using Invitrogen SuperScript III One-Step RT-PCR Platinum kit. Rat constant regions were amplified using IgG2b and kappa specific forward and reverse primers. PCR fragments were purified and subcloned into a plasmid for sequencing. DNA sequencing verified that the cloned sequences correspond to the rat IgG2b and kappa constant regions. The rat heavy and light chain constant regions were then subcloned into pCET expression vectors downstream from the mouse 8A9 variable heavy chain and light chain regions, respectively. These resulting vectors were expressed in CHO cells to produce mouse-rat 8A9 chimeric antibodies.

### Administration of Chimeric 8A9 Antibodies to HIP Rats

Animals.

50 male rats, strain CD(SD) HIP, 10-12 weeks of age, were used in the study.

Food and Water.

HIP rats were fed standard irradiated rodent chow ad libitum except during fasting; filtered drinking water was provided ad libitum.

Fasting.

Fasting of the HIP rats occurred every two weeks for assessment of fasting blood glucose levels. Food was removed 16-18 hours prior to the test and returned upon completion of the test.

Test Compound Preparation.

The test compounds consisted of mouse-rat chimeric 8A9 and PS2 antibodies (an isotype control). The test compounds were supplied as a sterile solution below 1 eu/mg endotoxin, and usually below <0.1 eu/mg endotoxin. The test compounds were formulated at pH 7.4 in 8 mM sodium phosphate, 2 mM potassium phosphate, 0.14M sodium chloride, and 10 mM potassium chloride.

Experimental Methodology.

HIP rats were divided into groups of 25 animals. Each group received test articles as shown in Table 1. The rats received weekly injections of the specified test articles, at 2 mL/kg (5 mg/mL) provided as a sterile solution. Injections were performed intraperitoneally (ip).

TABLE 1
Group Assignments

| | | | Test article | Dose | Dose | |
|---|---|---|---|---|---|---|
| Group | Strain | N | designation | mg/Kg | Volume Max. | Route |
| 1 | HIP | 25 | 8A9 | 10 | 2 ml/kg | ip |
| 2 | HIP | 25 | PS2 | 10 | 2 ml/kg | ip |

Body Weights.

Individual animal weights were determined twice weekly throughout the course of this study beginning with baseline weights on or prior to study day −7. As body weight is linked to the disease/phenotype being studied, a change in body weight was not automatically considered as an indicator of toxicity.

Glucose Screening.

Fasting glucose measurements were recorded every two weeks for each animal throughout the study via a handheld glucometer. Food was withheld the previous night and returned after blood has been taken via the tail vein. A pre-study glucose screen was conducted on or prior to Study day −7.

Sample Collections.

A sample of blood, not exceeding 0.5 mL, was taken via the tail vein every two weeks, processed to serum, and shipped to Elan and frozen at −70 C. A pre-study blood sample was drawn at Study Day −7 to establish baseline antibody level.

### Detection of Hemoglobin A1c

The level of hemoglobin A1c in the blood can be used to test for abnormal glucose metabolism. Hemoglobin A1c (HbA1c) was first recognized by Rahbar as an abnormal hemoglobin associated with diabetes in 1969. The abnormality was later identified as chemical glycation of the N-terminal lysine and valines of hemoglobin A. The chemical reaction includes an initial, reversible, formation of the aldehyde Schiff base, followed by essentially irreversible Amadori rearrangement to the stable ketoamine. See Saudek & Brick (2009), “The clinical use of hemoglobin A1c.” J Diabetes Sci Technol 3(4): 629-634.

The hemoglobin A1c levels in HIP rats were tested following 22 weeks of treatment with test articles according to Table 1 (above). Samples of blood were collected via the tail vein into tubes containing EDTA. The tubes were then inverted to allow for mixing and placed on ice. The samples were analyzed using an ACE Alera Analyzer. The analysis method was optimized for measuring rodents Hb A1c. Animals containing an anti-drug response to 8A9 were excluded from the evaluation.

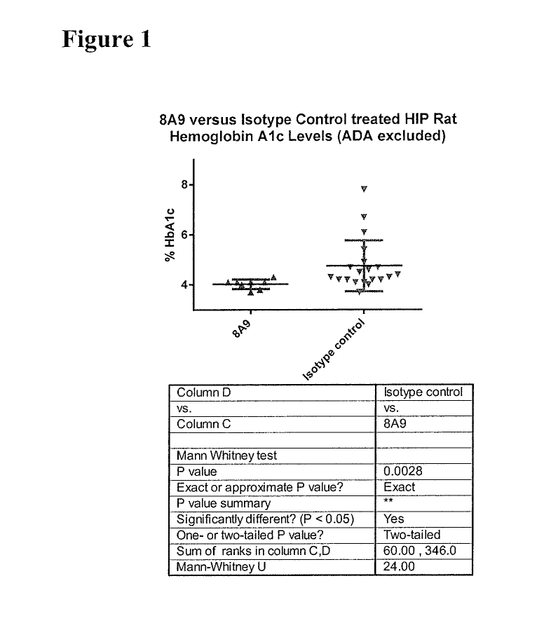

As shown in FIG. 1 and Table 2, HIP rats that had received the 8A9 antibody exhibited significantly lower hemoglobin A1c levels as compared to rats that had received the PS2 isotype control antibody (P=0.0028).

TABLE 2
Statistical Analysis for FIG. 1

| Column D vs. Column C | Isotype control vs. 8A9 |
|---|---|
| Mann Whitney test | |
| P value | 0.0028 |
| Exact or approximate P value? | Exact |
| P value summary | ** |
| Significantly different? (P < 0.05) | Yes |
| One- or two-tailed P value? | Two-tailed |
| Sum of ranks in column C, D | 60.00, 346.0 |
| Mann-Whitney U | 24.00 |

### Oral Glucose Tolerance Test

After 24 weeks of treatment with test articles as described in Table 1 (above), HIP rats were fasted for a minimum of 10 hours. After fasting, a draw of blood was taken and a baseline fasting blood glucose level was determined by hand held meter (Abbott Freedom Lite). No significant difference was observed in the average fasting blood glucose level of HIP rats treated with 8A9 antibody as compared to the average level in HIP rats that received the PS2 isotype control antibody.

Animals were then administered an oral dose of glucose at a dose level of 2 g/kg. Blood samples were collected at 30, 60, 90, 120, and 180 minutes after dosage, and blood glucose levels were detected. The standard time point for screening for glucose impairment is at 120 minutes.

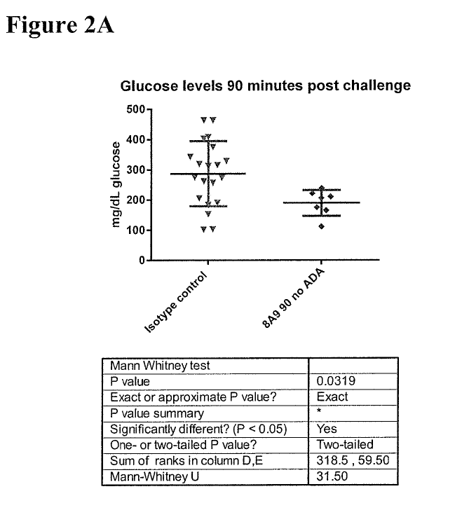

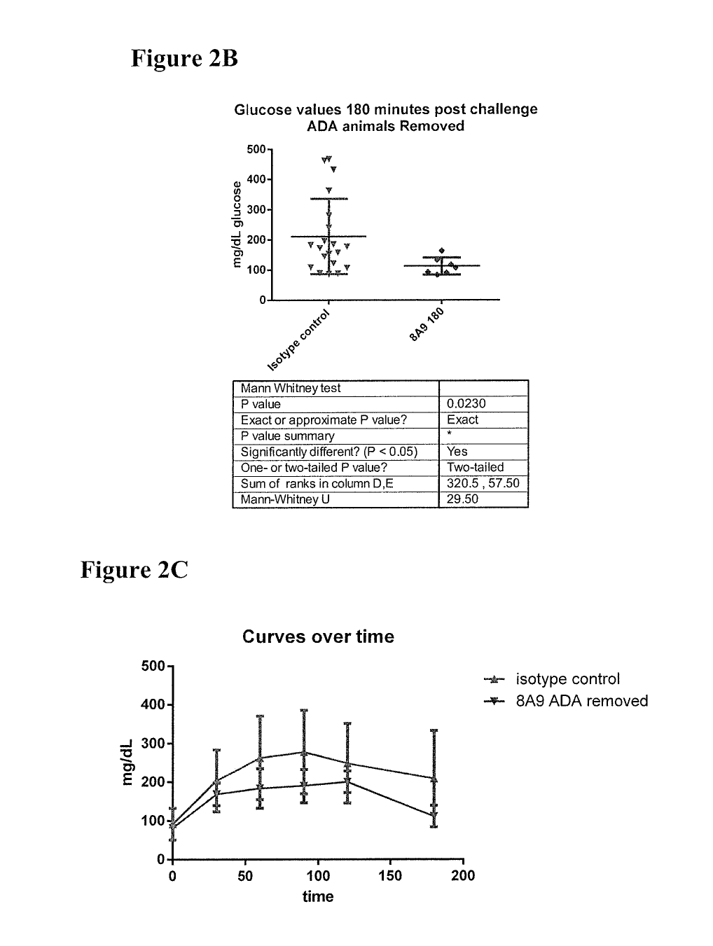

At the 90 and 180 minute post-glucose administration time points, HIP rats that received the 8A9 antibody exhibited a lower average blood glucose level than HIP rats that received the PS2 isotype control antibody. See FIGS. 2A and 2B and corresponding Tables 3 and 4. The differences were statistically significant (P=0.0319 and P=0.0230, respectively). At 30, 60, and 120 minutes after dosage, the average blood glucose levels in HIP rats receiving the 8A9 antibody were also lower than the average blood glucose levels in HIP rats that received the PS2 isotype control antibody, although the differences were not statistically significant. Overall, the data showed a clear trend toward lower blood glucose levels following glucose challenge in HIP rats that received the 8A9 antibody. See FIG. 2C.

TABLE 3
Statistical Analysis for FIG. 2A

| Column E vs. | 8A9 ADA removed vs. |
|---|---|
| Column D | Isotype control 90 |
| Mann Whitney test | |
| P value | 0.0319 |
| Exact or approximate P value? | Exact |
| P value summary | * |
| Significantly different? (P < 0.05) | Yes |
| One- or two-tailed P value? | Two-tailed |
| Sum of ranks in column D, E | 318.5, 59.50 |
| Mann-Whitney U | 31.50 |

TABLE 4
Statistical Analysis for FIG. 2B

| Column E vs. | 8A9 ADA removed vs. |
|---|---|
| Column D | Isotype control 180 |
| Mann Whitney test | |
| P value | 0.0230 |
| Exact or approximate P value? | Exact |
| P value summary | * |
| Significantly different? (P < 0.05) | Yes |
| One- or two-tailed P value? | Two-tailed |
| Sum of ranks in column D, E | 320.5, 57.50 |
| Mann-Whitney U | 29.50 |

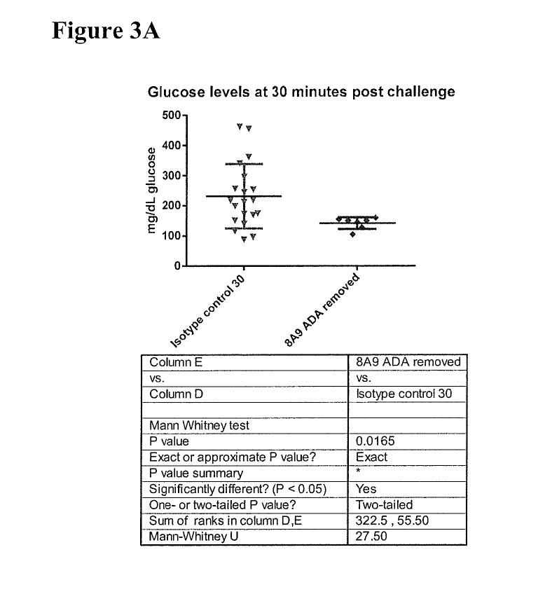

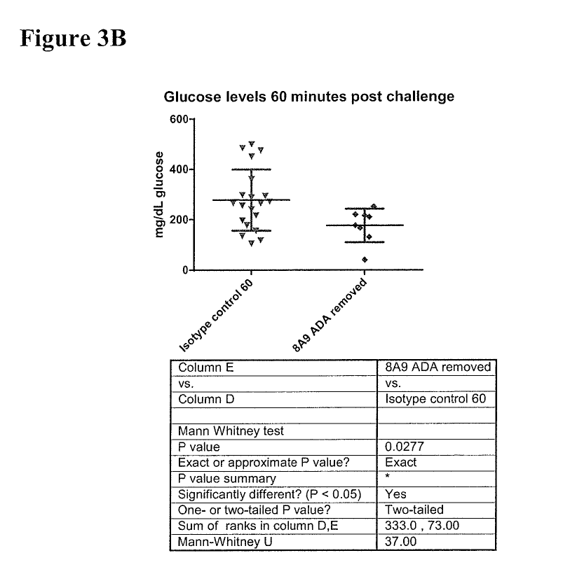

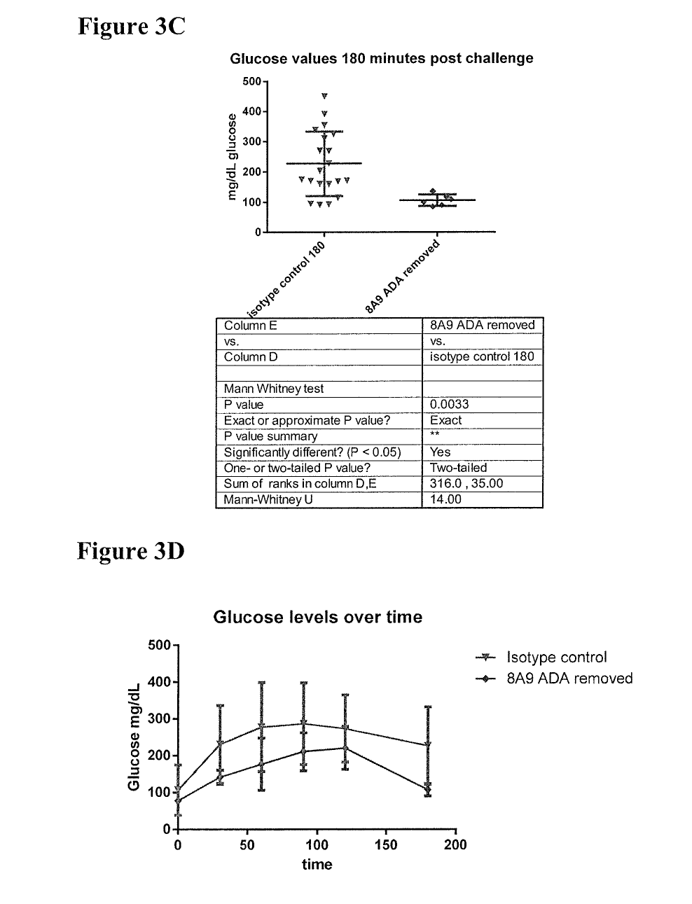

Following 28 weeks of treatment, the 8A9-treated and PS2 isotype control-treated HIP rats were again submitted to an oral glucose tolerance test, with blood glucose levels determined prior to glucose administration and at 30, 60, 90, 120, and 180 minutes into the test. Average fasting blood glucose levels were not significantly lower in rats treated with the 8A9 antibody. However, at 30, 60, and 180 minutes after glucose administration, the average blood glucose levels in HIP rats treated with the 8A9 antibody were significantly lower than the average blood glucose levels in HIP rats receiving the PS2 isotype control. See FIGS. 3A-3C and corresponding Tables 5-7 (at 30 min. p=0.0165; at 60 min. p=0.0277; and at 180 min. p=0.0033). In addition, average blood glucose levels in HIP rats treated with the 8A9 antibody were also lower at the 90 and 120 minute time points, though the difference was not statistically significant. As with the data obtained following 24 weeks of treatment, the 28 week data revealing a trend of lower glucose values and much tighter standard deviations in 8A9 treated rats. See FIG. 3D.

TABLE 5
Statistical Analysis for FIG. 3A

| Column E vs. | 8A9 ADA removed vs. |
|---|---|
| Column D | Isotype control 30 |
| Mann Whitney test | |
| P value | 0.0165 |
| Exact or approximate P value? | Exact |
| P value summary | * |
| Significantly different? (P < 0.05) | Yes |
| One- or two-tailed P value? | Two-tailed |
| Sum of ranks in column D, E | 322.5, 55.50 |
| Mann-Whitney U | 27.50 |

TABLE 6
Statistical Analysis for FIG. 3B

| Column E vs. | 8A9 ADA removed vs. |
|---|---|
| Column D | Isotype control 60 |
| Mann Whitney test | |
| P value | 0.0277 |
| Exact or approximate P value? | Exact |
| P value summary | * |
| Significantly different? (P < 0.05) | Yes |
| One- or two-tailed P value? | Two-tailed |
| Sum of ranks in column D, E | 333.0, 73.00 |
| Mann-Whitney U | 37.00 |

TABLE 7
Statistical Analysis for FIG. 3C

| Column E vs. | 8A9 ADA removed vs. |
|---|---|
| Column D | Isotype control 180 |
| Mann Whitney test | |
| P value | 0.0033 |
| Exact or approximate P value? | Exact |
| P value summary | ** |
| Significantly different? (P < 0.05) | Yes |
| One- or two-tailed P value? | Two-tailed |
| Sum of ranks in column D, E | 316.0, 35.00 |
| Mann-Whitney U | 14.00 |

Taken together, the blood glucose data obtained from HIP rats at 22, 24, and 28 weeks show that the 8A9 antibody affects multiple parameters implicated as indicators of the development of Type 2 diabetes, including Hemoglobin A1c levels, which increase based on the amount of time an animal has heighten circulating glucose levels. The 8A9 antibody also significantly helps maintain homeostasis of glucose levels after the animal is orally challenged with glucose.

### Example 3

### 8A9 Significantly Decreases IAPP Amyloid in the Pancreas and has a Trend Toward Improved Beta Cell Survival

The HIP rats from Example 2 were sacrificed at approximately 9 months of age and 30 weeks of treatment. The pancreas was removed from the test rats, fixed, and paraffin embedded.

Serial pancreas sections were deparaffinized and stained with a mouse monoclonal antibody to insulin (0.1 ug/mL; product number: 12018; Sigma-Aldrich, St. Louis, Mo.) on a Leica Bond Rx (Leica Microsystems Inc, Buffalo Groove, Ill.).

Insulin-positive beta cells were visualized with a 3,3′-diaminobenzidine tetrahydrochloride (DAB) chromogen, which produces a brown precipitate.

A modified thioflavine T staining protocol was employed to visualize deposited amyloid within the islets. Thioflavine T positive staining showed green fluorescence when imaged with a fluorescein isothiocyanate filter (excitation at 488 nm).

All slides were imaged using the Nanozoomer 2.0 HT (Hamamatsu Corp, Bridgewater, N.J.) equipped with 20× N.A. 0.75 objective (Olympus; Center Valley, Pa.) and a Sedat Quad filter set (Chroma Technology Corp; Bellows Falls, Vt.). The amounts of beta cells and deposited amyloid were determined by measuring the insulin- and thioflavine T-positive areas, respectively. Measurements are reported as values of the percent area of stained tissue over the total area of the entire pancreas section. Image analysis was performed on all the islets from one pancreas section per animal.

### Results

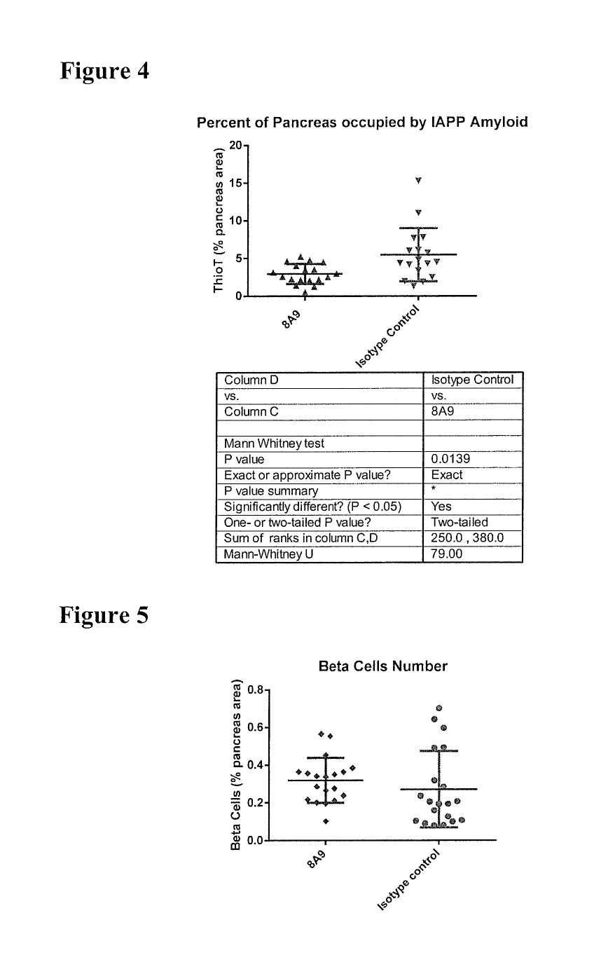

Evaluation of 8A9 was complicated by the high number of animals that developed an anti-drug response starting at week eight of treatment. Even so, a significant decrease in amyloid levels in the pancreas was detected. See FIG. 4 and Table 8. In addition, though not significant (P=0.069), a trend towards increased Beta-cell number was observed. See FIG. 5.

TABLE 8
Statistical Analysis for FIG. 4

| Column D vs. Column C | Isotype control vs. 8A9 |
|---|---|
| Mann Whitney test | |
| P value | 0.0139 |
| Exact or approximate P value? | Exact |
| P value summary | * |
| Significantly different? (P < 0.05) | Yes |
| One- or two-tailed P value? | Two-tailed |
| Sum of ranks in column C, D | 250.0, 380.0 |
| Mann-Whitney U | 79.00 |

### Example 4

### Humanization of 8A9

RNA was extracted from pelleted cells expressing the 8A9 antibody. The resulting RNA was reverse transcribed to produce cDNA, and nucleic acid sequences coding for the immunoglobulin heavy chain and light chain variable regions of the 8A9 antibody were amplified by PCR. The PCR products were gel purified, cloned, and sequenced.

Nucleic acid encoding the 8A9 heavy chain variable region has the sequence of SEQ ID NO: 3. The corresponding protein sequence, which includes a signal peptide at positions 1-19 (underlined) is as follows:

(SEQ ID NO: 4)
MAVLVLFLCLVAFPSCVLSQVQLKESGPGLVAPSQSLSITCTVSGFSLTN
YAVHWVRQPPGKGLEWLGVIWSGGNTNYNSALMSRLRISKDNSQSQVFLK
MNSLQTDDTGMYYCARDRGYYGGIYGFANWGQGTLVTVSS

Nucleic acid encoding the 8A9 light chain variable region has the sequence of SEQ ID NO: 5. The corresponding protein sequence, which includes a signal peptide at positions 1-20 (underlined) is as follows:

(SEQ ID NO: 6)
MRVPAHVFGFLLLWFPGTRCDIQMTQSPSSLSASLGKRVSLTCRASQEIS
GYLSWLQQKPDGTIKRLIYAASTSDSGVPKRFSGSRSGSDYSLTISSVES
EDFADYYCLQYADYPYTFGGGTKLEIK

The amino acid sequence for the mature 8A9 heavy chain variable region (SEQ ID NO: 7) is shown in Table 9, and the corresponding amino acid sequence for the mature 8A9 light chain variable region (SEQ ID NO: 29) is shown in Table 10. Kabat numbering is used throughout.

The 8A9 light chain variable region is a variable kappa (Vk) region that belongs to mouse Kabat subgroup 5, which corresponds to human Kabat subgroup 1. The 8A9 heavy chain variable region belongs to mouse Kabat subgroup 1b, which corresponds to human Kabat subgroup 2. See Kabat et al. (1991) Sequences of Proteins of Immunological Interest, Fifth Edition. NIH Publication No. 91-3242.

Analysis of the CDRs of the 8A9 Vk region reveals: a 11 residue CDR-L1 (SEQ ID NO: 30) belonging to canonical class 2; a 7 residue CDR-L2 (SEQ ID NO: 31) belonging to canonical class 1; and a 9 residue CDR-L3 (SEQ ID NO: 32) belonging to canonical class 1. See Martin & Thornton (1996), Structural families in loops of homologous proteins: automatic classification, modeling and application to antibodies. J Mol Biol. 263:800-15. Similar analysis of the CDRs of the 8A9 Vh region reveals: a 10 residue CDR-H1 (SEQ ID NO: 8) belonging to canonical class 1; and a 16 residue CDR-H2 (SEQ ID NO: 9) belonging to canonical class 1. See Martin & Thornton (1996), supra. The 8A9 Vh region also includes a 13 residue CDR-H3 (SEQ ID NO: 10) that does not belong to any canonical class. The shading in Tables 9 and 10 identify the location of the CDR residues. The CDR-H1 residues identified in Table 9 are a composite of the CDR-H1 residues as identified according to Kabat and Chothia. The CDR-H2 and CDR-H3 identified in Table 9 and the CDR-L1, CDR-L2, and CDR-L3 residues identified in Table 10 all correspond to the CDR residues as identified according to Kabat.

A search was made over the protein sequences in the PDB database to identify structures that could provide a rough model of 8A9. See Deshpande et al. (2005), The RCSB Protein Data Bank: a redesigned query system and relational database based on the mmCIF schema, Nucleic Acids Res. 33:D233-7. The crystal structure of germline FAB 48G7 (pdb code 2RCS; Wedemayer et al. (1997), Structural Insights into the Evolution of an Antibody Combining Site, Science 276:1665) was settled upon for the Vk structure since it has good resolution (2.1A) and overall sequence similarity with 8A9 Vk, retaining the same canonical structures for the loops. The sequence of the mature light chain variable region of FAB 48G7 is shown in SEQ ID NO: 33. The heavy chain of the 13G5 antibody (pdb code 2GJZ; Muller et al. (2007), Bifunctional Catalysis of Proton Transfer at an Antibody Active Site, J. Am. Chem. Soc. 129:460-61) was selected to model the Vh structure since it has reasonably good resolution (2.65A) and the same canonical structures for CDR-H1 and CDR-H2. The sequence of the mature heavy chain variable region of 13G5 is shown in SEQ ID NO: 11. Bioluminate/Maestro software was used to model a rough structure of 8A9 Fv.

Analysis of the residues at the interface between the 8A9 Vk and Vh regions revealed only commonly found residues at the interface, except for (1) the leucine at position L36, which is typically occupied by Y or F, and (2) the isoleucine at position L44, which is typically occupied by P. These atypical residues are prime targets for back-mutation.

A search of the non-redundant protein sequence database from NCBI allowed selection of suitable human frameworks into which to graft the 8A9 murine CDRs. For Vk, a human kappa light chain with NCBI accession code AAZ09096.1 (GI:70798773; Stamatopoulos et al. (2005); SEQ ID NO: 34) was chosen. This human Vk region has the same canonical classes for CDR-L1 and CDR-L2 as the 8A9 Vk region and is a member of the Kabat human kappa subgroup 1. For Vh, a human Ig heavy chain with NCBI accession code BAC02410 (GI:21670801; Akahori et al. (2002); SEQ ID NO: 12) was chosen. It includes the same canonical structures for CDR-H1 and CDR-H2 and is a member of Kabat human heavy subgroup 2.

The humanized heavy and light chain variable region designs and backmutations (lowercase) based on the selected human frameworks are shown in Table 9 and Table 10, respectively.

### Humanized Vh Region Designs

Eight different humanized versions of the 8A9 Vh region were designed, H1 through H7 and H7b. For backmutations, residues H16, H17, H46, H74, and H82C were backmutated in all eight humanized versions to Q, S, E, S, and L, since these residues appear more frequently in human framework sequences. Residues H37, H48, H69, H71, H73, H75, H78, H79, and H85 were then focused on for additional backmutations. In versions H1 (SEQ ID NO: 13), H3 (SEQ ID NO: 15), H4 (SEQ ID NO: 16), H6 (SEQ ID NO: 18), H7 (SEQ ID NO: 19), and H7b (SEQ ID NO: 20) H37 was backmutated to V (I37V), since this is an interface residue and valine is slightly more frequent than isoleucine; H69 was backmutated to isoleucine (M66I), since this is a Vernier residue and methionine is rare in this position; and H71 was backmutated to lysine (V68K), since this is a Vernier residue and lysine is much bulkier and more basic as compared to valine. In versions H1 (SEQ ID NO: 13), H7 (SEQ ID NO: 19), and H7b (SEQ ID NO: 20) H48 was backmutated to leucine (I48L), since it is a canonical/CDR interacting residue; H73 was backmutated to asparagine (T7ON), since this is a canonical/CDR contact residue; and H75 was backmutated to glutamine, since this is a Vernier residue. In versions H4 (SEQ ID NO: 16), H5 (SEQ ID NO: 17), H6 (SEQ ID NO: 18), H7 (SEQ ID NO: 19), and H7b (SEQ ID NO: 20) H78 was backmutated to valine (F75V), since this residue appears to pack under a CDR. In versions H4 (SEQ ID NO: 16), H7 (SEQ ID NO: 19), and H7b (SEQ ID NO: 20) H79 was backmutated to phenylalanine (S76F), since this residue also seems to pack under a CDR. In versions H2 (SEQ ID NO: 14), H3 (SEQ ID NO: 15), H4 (SEQ ID NO: 16), H5 (SEQ ID NO: 17), and H6 (SEQ ID NO: 18) H85 was backmutated to aspartic acid (A85D), since aspartic acid is more frequent at this position in human framework sequences. Version H7b (SEQ ID NO: 20) was further mutated at H1 to glutamate (Q1E) to reduce pyroglutamate formation. Position H1 is the only residue that differs between H7b and H7.

TABLE 9
Humanized 8A9 Region Designs

| | | | Hu VH | | | | | | | | |
|---|---|---|---|---|---|---|---|---|---|---|---|
| | | Murine | Acptr | | | | | | | | |
| Kabat | | 8A9 | FR | H1 | H2 | H3 | H4 | H5 | H6 | H7 | H7b |
| residue | FR or | SEQ ID | SEQ ID | SEQ ID | SEQ ID | SEQ ID | SEQ ID | SEQ ID | SEQ ID | SEQ ID | SEQ ID |
| # | CDR | NO: 7 | NO: 12 | NO: 13 | NO: 14 | NO: 15 | NO: 16 | NO: 17 | NO: 18 | NO: 19 | NO: 20 |
| 1 | Fr1 | Q | Q | Q | Q | Q | Q | Q | Q | Q | E |
| 2 | Fr1 | V | V | V | V | V | V | V | V | V | V |
| 3 | Fr1 | Q | Q | Q | Q | Q | Q | Q | Q | Q | Q |
| 4 | Fr1 | L | L | L | L | L | L | L | L | L | L |
| 5 | Fr1 | K | Q | Q | Q | Q | Q | Q | Q | Q | Q |
| 6 | Fr1 | E | Q | Q | Q | Q | Q | Q | Q | Q | Q |
| 7 | Fr1 | S | S | S | S | S | S | S | S | S | S |
| 8 | Fr1 | G | G | G | G | G | G | G | G | G | G |
| 9 | Fr1 | P | P | P | P | P | P | P | P | P | P |
| 10 | Fr1 | G | G | G | G | G | G | G | G | G | G |
| 11 | Fr1 | L | L | L | L | L | L | L | L | L | L |
| 12 | Fr1 | V | V | V | V | V | V | V | V | V | V |
| 13 | Fr1 | A | K | K | K | K | K | K | K | K | K |
| 14 | Fr1 | P | P | P | P | P | P | P | P | P | P |
| 15 | Fr1 | S | S | S | S | S | S | S | S | S | S |
| 16 | Fr1 | Q | E | Q | Q | Q | Q | Q | Q | Q | Q |
| 17 | Fr1 | S | T | S | S | S | S | S | S | S | S |
| 18 | Fr1 | L | L | L | L | L | L | L | L | L | L |
| 19 | Fr1 | S | S | S | S | S | S | S | S | S | S |
| 20 | Fr1 | I | L | L | L | L | L | L | L | L | L |
| 21 | Fr1 | T | T | T | T | T | T | T | T | T | T |
| 22 | Fr1 | C | C | C | C | C | C | C | C | C | C |
| 23 | Fr1 | T | T | T | T | T | T | T | T | T | T |
| 24 | Fr1 | V | V | V | V | V | V | V | V | V | V |
| 25 | Fr1 | S | S | S | S | S | S | S | S | S | S |
| 26 | CDR-H1 | G | G | G | G | G | G | G | G | G | G |
| 27 | CDR-H1 | F | G | F | F | F | F | F | F | F | F |
| 28 | CDR-H1 | S | S | S | S | S | S | S | S | S | S |
| 29 | CDR-H1 | L | L | L | L | L | L | L | L | L | L |
| 30 | CDR-H1 | T | T | T | T | T | T | T | T | T | T |
| 31 | CDR-H1 | N | N | N | N | N | N | N | N | N | N |
| 32 | CDR-H1 | Y | Y | Y | Y | Y | Y | Y | Y | Y | Y |
| 33 | CDR-H1 | A | Y | A | A | A | A | A | A | A | A |
| 34 | CDR-H1 | V | W | V | V | V | V | V | V | V | V |
| 35 | CDR-H1 | H | S | H | H | H | H | H | H | H | H |
| 35A | CDR-H1 | — | — | — | — | — | — | — | — | — | — |
| 35B | CDR-H1 | — | — | — | — | — | — | — | — | — | — |
| 36 | Fr2 | W | W | W | W | W | W | W | W | W | W |
| 37 | Fr2 | V | I | V | I | V | V | I | V | V | V |
| 38 | Fr2 | R | R | R | R | R | R | R | R | R | R |
| 39 | Fr2 | Q | Q | Q | Q | Q | Q | Q | Q | Q | Q |
| 40 | Fr2 | P | P | P | P | P | P | P | P | P | P |
| 41 | Fr2 | P | P | P | P | P | P | P | P | P | P |
| 42 | Fr2 | G | G | G | G | G | G | G | G | G | G |
| 43 | Fr2 | K | K | K | K | K | K | K | K | K | K |
| 44 | Fr2 | G | G | G | G | G | G | G | G | G | G |
| 45 | Fr2 | L | L | L | L | L | L | L | L | L | L |
| 46 | Fr2 | E | Q | E | E | E | E | E | E | E | E |
| 47 | Fr2 | W | W | W | W | W | W | W | W | W | W |
| 48 | Fr2 | L | I | L | I | I | I | I | I | L | L |
| 49 | Fr2 | G | G | G | G | G | G | G | G | G | G |
| 50 | CDR-H2 | V | Y | V | V | V | V | V | V | V | V |
| 51 | CDR-H2 | I | I | I | I | I | I | I | I | I | I |
| 52 | CDR-H2 | W | S | W | W | W | W | W | W | W | W |
| 52A | CDR-H2 | — | — | — | — | — | — | — | — | — | — |
| 52B | CDR-H2 | — | — | — | — | — | — | — | — | — | — |
| 52C | CDR-H2 | — | — | — | — | — | — | — | — | — | — |
| 53 | CDR-H2 | S | H | S | S | S | S | S | S | S | S |
| 54 | CDR-H2 | G | S | G | G | G | G | G | G | G | G |
| 55 | CDR-H2 | G | G | G | G | G | G | G | G | G | G |
| 56 | CDR-H2 | N | I | N | N | N | N | N | N | N | N |
| 57 | CDR-H2 | T | T | T | T | T | T | T | T | T | T |
| 58 | CDR-H2 | N | N | N | N | N | N | N | N | N | N |
| 59 | CDR-H2 | Y | Y | Y | Y | Y | Y | Y | Y | Y | Y |
| 60 | CDR-H2 | N | N | N | N | N | N | N | N | N | N |
| 61 | CDR-H2 | S | P | S | S | S | S | S | S | S | S |
| 62 | CDR-H2 | A | S | A | A | A | A | A | A | A | A |
| 63 | CDR-H2 | L | L | L | L | L | L | L | L | L | L |
| 64 | CDR-H2 | M | M | M | M | M | M | M | M | M | M |
| 65 | CDR-H2 | S | S | S | S | S | S | S | S | S | S |
| 66 | Fr3 | R | R | R | R | R | R | R | R | R | R |
| 67 | Fr3 | L | L | L | L | L | L | L | L | L | L |
| 68 | Fr3 | R | T | T | T | T | T | T | T | T | T |
| 69 | Fr3 | I | M | I | M | I | I | M | I | I | I |
| 70 | Fr3 | S | S | S | S | S | S | S | S | S | S |
| 71 | Fr3 | K | V | K | V | K | K | V | K | K | K |
| 72 | Fr3 | D | D | D | D | D | D | D | D | D | D |
| 73 | Fr3 | N | T | N | T | T | T | T | T | N | N |
| 74 | Fr3 | S | P | S | S | S | S | S | S | S | S |
| 75 | Fr3 | Q | K | Q | K | K | K | K | K | Q | Q |
| 76 | Fr3 | S | N | N | N | N | N | N | N | N | N |
| 77 | Fr3 | Q | Q | Q | Q | Q | Q | Q | Q | Q | Q |
| 78 | Fr3 | V | F | F | F | F | V | V | V | V | V |
| 79 | Fr3 | F | S | S | S | S | F | S | S | F | F |
| 80 | Fr3 | L | L | L | L | L | L | L | L | L | L |
| 81 | Fr3 | K | K | K | K | K | K | K | K | K | K |
| 82 | Fr3 | M | L | L | L | L | L | L | L | L | L |
| 82A | Fr3 | N | N | N | N | N | N | N | N | N | N |
| 82B | Fr3 | S | S | S | S | S | S | S | S | S | S |
| 82C | Fr3 | L | V | L | L | L | L | L | L | L | L |
| 83 | Fr3 | Q | T | T | T | T | T | T | T | T | T |
| 84 | Fr3 | T | T | T | T | T | T | T | T | T | T |
| 85 | Fr3 | D | A | A | D | D | D | D | D | A | A |
| 86 | Fr3 | D | D | D | D | D | D | D | D | D | D |
| 87 | Fr3 | T | T | T | T | T | T | T | T | T | T |
| 88 | Fr3 | G | G | G | G | G | G | G | G | G | G |
| 89 | Fr3 | M | V | V | V | V | V | V | V | V | V |
| 90 | Fr3 | Y | Y | Y | Y | Y | Y | Y | Y | Y | Y |
| 91 | Fr3 | Y | Y | Y | Y | Y | Y | Y | Y | Y | Y |
| 92 | Fr3 | C | C | C | C | C | C | C | C | C | C |
| 93 | Fr3 | A | A | A | A | A | A | A | A | A | A |
| 94 | Fr3 | R | R | R | R | R | R | R | R | R | R |
| 95 | CDR-H3 | D | E | D | D | D | D | D | D | D | D |
| 96 | CDR-H3 | R | G | R | R | R | R | R | R | R | R |
| 97 | CDR-H3 | G | V | G | G | G | G | G | G | G | G |
| 98 | CDR-H3 | Y | L | Y | Y | Y | Y | Y | Y | Y | Y |
| 99 | CDR-H3 | Y | P | Y | Y | Y | Y | Y | Y | Y | Y |
| 100 | CDR-H3 | G | A | G | G | G | G | G | G | G | G |
| 100A | CDR-H3 | G | S | G | G | G | G | G | G | G | G |
| 100B | CDR-H3 | I | I | I | I | I | I | I | I | I | I |
| 103 | Fr4 | W | W | W | W | W | W | W | W | W | W |
| 104 | Fr4 | G | G | G | G | G | G | G | G | G | G |
| 105 | Fr4 | Q | Q | Q | Q | Q | Q | Q | Q | Q | Q |
| 106 | Fr4 | G | G | G | G | G | G | G | G | G | G |
| 107 | Fr4 | T | T | T | T | T | T | T | T | T | T |
| 108 | Fr4 | L | L | L | L | L | L | L | L | L | L |
| 109 | Fr4 | V | V | V | V | V | V | V | V | V | V |
| 110 | Fr4 | T | T | T | T | T | T | T | T | T | T |
| 111 | Fr4 | V | V | V | V | V | V | V | V | V | V |
| 112 | Fr4 | S | S | S | S | S | S | S | S | S | S |
| 113 | Fr4 | S | S | S | S | S | S | S | S | S | S |

Exemplary nucleic acid sequences encoding humanized 8A9 H1 through H7 and H7b are provided in SEQ ID NOs: 21 through 27 and 28, respectively. For the glutamate residue at position H1 of version H7b, two different codons were considered for the coding sequence, GAG and GAA. See SEQ ID NO: 28. The GAA codon was used in the final design of the H7b coding sequence, but GAG could have been used as well.

### Humanized Vk Region Designs

Three different humanized versions of the 8A9 Vk region were designed, L1, L2, and L3. For backmutations, residues L36, L44, L46, L66, L69, L71, and L85 were ultimately focused on. L36 (Y36L) is a heavy chain/light chain packing residue; L44 (P44I) is a heavy chain/light chain packing residue; L46 (L46R) is a Vernier residue that provides foundation for CDR-L3; L66 (G66R) is a Vernier residue that provides foundation for CDR-L1; L69 (T69S) is a Vernier residue that packs beneath CDR-L1; L71 (F71Y) is a canonical CDR interacting residue; and L85 (T85D) provides for potential interference (based on the homology model) with tyrosine at L87 when occupied by threonine. In version L1 (SEQ ID NO: 35), L26, L44, L46, L66, L69, and L71 are backmutated to L, I, R, R, S, and Y, respectively. In version L2 (SEQ ID NO: 36), there are no backmutations. Version L3 (SEQ ID NO: 37) includes all of the backmutations of L1 plus the backmutation at L85 (T85D).

TABLE 10
Humanized 8A9 Vk Regions

| | FR | | Hu VL | | | |
|---|---|---|---|---|---|---|
| Kabat | or | Murine 8A9 | Acceptor Fr | L1 | L2 | L3 |
| residue # | CDR | SEQ ID NO: 29 | SEQ ID NO: 34 | SEQ ID NO: 35 | SEQ ID NO: 36 | SEQ ID NO: 37 |
| 1 | Fr1 | D | D | D | D | D |
| 2 | Fr1 | I | I | I | I | I |
| 3 | Fr1 | Q | Q | Q | Q | Q |
| 4 | Fr1 | M | M | M | M | M |
| 5 | Fr1 | T | T | T | T | T |
| 6 | Fr1 | Q | Q | Q | Q | Q |
| 7 | Fr1 | S | S | S | S | S |
| 8 | Fr1 | P | P | P | P | P |
| 9 | Fr1 | S | S | S | S | S |
| 10 | Fr1 | S | S | S | S | S |
| 11 | Fr1 | L | L | L | L | L |
| 12 | Fr1 | S | S | S | S | S |
| 13 | Fr1 | A | A | A | A | A |
| 14 | Fr1 | S | S | S | S | S |
| 15 | Fr1 | L | V | V | V | V |
| 16 | Fr1 | G | G | G | G | G |
| 17 | Fr1 | K | D | D | D | D |
| 18 | Fr1 | R | R | R | R | R |
| 19 | Fr1 | V | V | V | V | V |
| 20 | Fr1 | S | T | T | T | T |
| 21 | Fr1 | L | I | I | I | I |
| 22 | Fr1 | T | T | T | T | T |
| 23 | Fr1 | C | C | C | C | C |
| 24 | CDR-L1 | R | R | R | R | R |
| 25 | CDR-L1 | A | A | A | A | A |
| 26 | CDR-L1 | S | S | S | S | S |
| 27 | CDR-L1 | Q | Q | Q | Q | Q |
| 27A | CDR-L1 | — | — | — | — | — |
| 27B | CDR-L1 | — | — | — | — | — |
| 27C | CDR-L1 | — | — | — | — | — |
| 27D | CDR-L1 | — | — | — | — | — |
| 27E | CDR-L1 | — | — | — | — | — |
| 27F | CDR-L1 | — | — | — | — | — |
| 28 | CDR-L1 | E | S | E | E | E |
| 29 | CDR-L1 | I | I | I | I | I |
| 30 | CDR-L1 | S | S | S | S | S |
| 31 | CDR-L1 | G | S | G | G | G |
| 32 | CDR-L1 | Y | Y | Y | Y | Y |
| 33 | CDR-L1 | L | L | L | L | L |
| 34 | CDR-L1 | S | N | S | S | S |
| 35 | Fr2 | W | W | W | W | W |
| 36 | Fr2 | L | Y | L | Y | L |
| 37 | Fr2 | Q | Q | Q | Q | Q |
| 38 | Fr2 | Q | Q | Q | Q | Q |
| 39 | Fr2 | K | K | K | K | K |
| 40 | Fr2 | P | P | P | P | P |
| 41 | Fr2 | D | G | G | G | G |
| 42 | Fr2 | G | K | K | K | K |
| 43 | Fr2 | T | A | A | A | A |
| 44 | Fr2 | I | P | I | P | I |
| 45 | Fr2 | K | K | K | K | K |
| 46 | Fr2 | R | L | R | L | R |
| 47 | Fr2 | L | L | L | L | L |
| 48 | Fr2 | I | I | I | I | I |
| 49 | Fr2 | Y | Y | Y | Y | Y |
| 50 | CDR-L2 | A | A | A | A | A |
| 51 | CDR-L2 | A | A | A | A | A |
| 52 | CDR-L2 | S | S | S | S | S |
| 53 | CDR-L2 | T | S | T | T | T |
| 54 | CDR-L2 | S | L | S | S | S |
| 55 | CDR-L2 | D | Q | D | D | D |
| 56 | CDR-L2 | S | S | S | S | S |
| 57 | Fr3 | G | G | G | G | G |
| 58 | Fr3 | V | V | V | V | V |
| 59 | Fr3 | P | P | P | P | P |
| 60 | Fr3 | K | S | S | S | S |
| 61 | Fr3 | R | R | R | R | R |
| 62 | Fr3 | F | F | F | F | F |
| 63 | Fr3 | S | S | S | S | S |
| 64 | Fr3 | G | G | G | G | G |
| 65 | Fr3 | S | S | S | S | S |
| 66 | Fr3 | R | G | R | G | R |
| 67 | Fr3 | S | S | S | S | S |
| 68 | Fr3 | G | G | G | G | G |
| 69 | Fr3 | S | T | S | T | S |
| 70 | Fr3 | D | D | D | D | D |
| 71 | Fr3 | Y | F | Y | F | Y |
| 72 | Fr3 | S | T | T | T | T |
| 73 | Fr3 | L | L | L | L | L |
| 74 | Fr3 | T | T | T | T | T |
| 75 | Fr3 | I | I | I | I | I |
| 76 | Fr3 | S | S | S | S | S |
| 77 | Fr3 | S | S | S | S | S |
| 78 | Fr3 | V | L | L | L | L |
| 79 | Fr3 | E | Q | Q | Q | Q |
| 80 | Fr3 | S | P | P | P | P |
| 81 | Fr3 | E | E | E | E | E |
| 82 | Fr3 | D | D | D | D | D |
| 83 | Fr3 | F | F | F | F | F |
| 84 | Fr3 | A | A | A | A | A |
| 85 | Fr3 | D | T | T | T | D |
| 86 | Fr3 | Y | Y | Y | Y | Y |
| 87 | Fr3 | Y | Y | Y | Y | Y |
| 88 | Fr3 | C | C | C | C | C |
| 89 | CDR-L3 | L | Q | L | L | L |
| 90 | CDR-L3 | Q | Q | Q | Q | Q |
| 91 | CDR-L3 | Y | S | Y | Y | Y |
| 92 | CDR-L3 | A | Y | A | A | A |
| 93 | CDR-L3 | D | S | D | D | D |
| 94 | CDR-L3 | Y | T | Y | Y | Y |
| 95 | CDR-L3 | P | P | P | P | P |
| 95A | CDR-L3 | — | — | — | — | — |
| 95B | CDR-L3 | — | — | — | — | — |
| 95C | CDR-L3 | — | — | — | — | — |
| 95D | CDR-L3 | — | — | — | — | — |
| 95E | CDR-L3 | — | — | — | — | — |
| 95F | CDR-L3 | — | — | — | — | — |
| 96 | CDR-L3 | Y | L | Y | Y | Y |
| 97 | CDR-L3 | T | T | T | T | T |
| 98 | Fr4 | F | F | F | F | F |
| 99 | Fr4 | G | G | G | G | G |
| 100 | Fr4 | G | G | G | G | G |
| 101 | Fr4 | G | G | G | G | G |
| 102 | Fr4 | T | T | T | T | T |
| 103 | Fr4 | K | K | K | K | K |
| 104 | Fr4 | L | V | V | V | V |
| 105 | Fr4 | E | E | E | E | E |
| 106 | Fr4 | I | I | I | I | I |
| 106A | Fr4 | — | — | — | — | — |
| 107 | Fr4 | K | K | K | K | K |

Exemplary nucleic acid sequences encoding humanized 8A9 L1, L2, and L3 are provided in SEQ ID NOs: 38, 39, and 40, respectively.

### Example 5

### IAPP-Binding Affinity of Humanized 8A9 Antibodies

The affinity of various combinations of 8A9 humanized heavy chains and humanized light chain proteins for hIAPP was analyzed on a Biacore instrument. Biacore analysis was performed by preparing an anti-human CM5 chip following the protocol provided in the kit. The 8A9 antibody was captured such that the maximum binding of rat IAPP would not exceed 20-30 RU. Rat IAPP was used in the assays instead of human IAPP because the epitope recognized by the 8A9 antibody is identical in rat and human IAPP and rat IAPP does not aggregate in solution, allowing for cleaner kinetics measurements. Various concentrations of rat IAPP were flowed over the sensor for a time long enough that at least the higher concentrations resulted in equilibrium binding, and then allowed to dissociate from the chip for a length of time such that at least 10% of total bound IAPP had dissociated. Data was blank subtracted to both an irrelevant sensor not containing 8A9 and 0 IAPP concentration to account for the dissociation of 8A9 from the anti-human capture. Data was then analyzed using a global 1:1 fit.

As shown in Table 11, the H7L1 version of humanized 8A9 antibody has an affinity for hIAPP that is only slightly lower than that of the murine 8A9 antibody (K_{D}=2.5 nM for H7L1 as compared to K_{D}=1.9 nM for murine 8A9) and exceeds the affinity of mouse-human chimeric 8A9 antibody (K_{D}=3.1 nM). In addition, the H7L3 version of humanized 8A9 antibody has an affinity for hIAPP that is within a factor of two of that of the murine 8A9 antibody (K_{D}=3.7 nM for H7L3 as compared to K_{D}=1.9 nM for murine 8A9) and only slightly less than the affinity of mouse-human chimeric 8A9 antibody (K_{D}=3.1 nM).

TABLE 11
Affinity of 8A9 Humanized Antibody Versions for hIAPP

| 8A9 | Mouse aa in Fwrk | | K_{D} | Kₒₙ | K_{off} |
|---|---|---|---|---|---|
| variant | HC | LC | nM | 1/Ms | 1/s |
| m8A9 | 82Fwrk/ | 80Fwrk/ | 1.9 | 1.6 × 10⁶/s | 1.9 × 10³/s |
| | 39CDR | 27CDR | | | |
| ch8A9 | 82Fwrk/ | 80Fwrk/ | 3.1 | 6.5 × 10⁵/s | 2.0 × 10³/s |
| | 39CDR | 27CDR | | | |
| H8A9- | 16Q, 17S, 37V, | 36L, 44I, | 2.5 | 7.6 × 10⁵/s | 1.9 × 10³/s |
| H7L1 | 46E, 48L, 69I, | 46R, 66R, | | | |
| | 71K, 73N, 74S, | 69S, 71Y | | | |
| | 75Q, 78V, 79F, | | | | |
| | and 82C-L | | | | |
| H8A9- | 16Q, 17S, 37V, | 36L, 44I, | 3.7 | 6.6 × 10⁵/s | 2.5 × 10³/s |
| H7L3 | 46E, 48L, 69I, | 46R, 66R, | | | |
| | 71K, 73N, 74S, | 69S, 71Y, | | | |
| | 75Q, 78V, 79F, | and 85D | | | |
| | and 82C-L | | | | |

Various changes in form and details can be made therein without departing from the spirit and scope of the invention. Unless otherwise apparent from the context, any embodiment, aspect, element, feature, step or the like can be used in combination with any other. Insofar as information associated with a citation may change with time, the information associated with the citation at the earliest effective filing date is meant, the earliest effective filing date for a citation meaning the filing date of the present application or earlier priority application disclosing the citation. All references, issued patents and patent applications cited within the body of the instant specification are hereby incorporated by reference in their entirety, for all purposes.

1. (canceled)

2. An isolated monoclonal antibody that competes with monoclonal antibody 8A9 for binding to human IAPP.

3-4. (canceled)

5. The isolated monoclonal antibody of claim 2 that binds to the same epitope as antibody 8A9.

6. The antibody of claim 5 that is chimeric, veneered, humanized or human.

7. The antibody of claim 9 comprising three light chain CDRs as shown in SEQ ID NOs: 30-32, respectively, and three heavy chain CDRs as shown in SEQ ID NOs: 8-10, respectively, of monoclonal antibody 8A9.

8. (canceled)

9. The antibody of claim 6, wherein the antibody is a humanized antibody.

10-14. (canceled)

15. The antibody of claim 7, wherein the antibody is a single-chain Fv antibody or a Fab fragment.

16. (canceled)

17. The antibody of claim 7, comprising a mature heavy chain variable region having an amino acid sequence at least 90% identical to H7 (SEQ ID NO: 19) and a mature light chain variable region having an amino acid sequence at least 90% identical to L1 (SEQ ID NO: 35), wherein the antibody specifically binds to human IAPP.

18. (canceled)

19. The antibody of claim 17, provided at least one of positions H1, H16, H17, H37, H46, H48, H69, H71, H73, H74, H75, H78, H79, H82C, and H85 in the heavy chain variable region (Kabat numbering) is occupied by E, Q, S, V, E, L, I, K, N, S, Q, V, F, L, and D, respectively.

20. The antibody of claim 19, provided positions H16, H17, H46, H48, H69, H71, H73, H74, H78, H79 and H82C are occupied by Q, S, E, L, I, K, N, S, V, F and L, respectively.

21-24. (canceled)

25. The antibody of any one of claims 19-20, provided positions L36, L44, L46, L66, L69, and L71 are occupied by L, I, R, R, S, and Y, respectively.

26. The antibody of claim 25, provided position L85 is occupied by D.

27. The antibody of claim 17, comprising a mature heavy chain variable region having an amino acid sequence at least 95% identical to H7 (SEQ ID NO: 19) and a mature light chain variable region at least 95% identical to L1 (SEQ ID NO: 35).

28. The antibody of claim 17, wherein the mature heavy chain variable region is fused to a heavy chain constant region and the mature light chain variable region is fused to a light chain constant region.

29. The antibody of claim 28, wherein the heavy chain constant region is a mutant form of a natural human heavy chain constant region which has reduced binding to an Fcγ receptor relative to the natural human heavy chain constant region.

30. The antibody of claim 28, wherein the heavy chain constant region is of IgG1 isotype.

31. The antibody of claim 28, wherein the mature heavy chain variable region is fused to a heavy chain constant region having the sequence of SEQ ID NO: 42 and/or the mature light chain variable region is fused to a light chain constant region having the sequence of SEQ ID NO: 44.

32. The antibody of claim 17, provided any differences in CDRs of the mature heavy chain variable region and mature light chain variable region from H7 and L1 (SEQ ID NOs: 19 and 35, respectively) reside in positions H60-H65.

33. The antibody of claim 17, wherein the mature heavy chain variable region has an amino acid sequence designated H7 (SEQ ID NO: 19) and the mature light chain variable region has an amino acid sequence designated L1 (SEQ ID NO: 35).

34. (canceled)

35. A pharmaceutical composition comprising an antibody as defined in claim 7 and a pharmaceutically-acceptable carrier.

36. A nucleic acid encoding the heavy and/or light chain(s) of an antibody as described in claim 7.

37. The nucleic acid of claim 36, wherein the heavy chain is encoded by the nucleic acid sequence of SEQ ID NO: 27 and the light chain is encoded by the nucleic acid sequence of SEQ ID NO: 38.

38. A recombinant expression vector comprising a nucleic acid according to claim 36.

39. A host cell transformed with the recombinant expression vector according to claim 38.

40. A method of humanizing an antibody, comprising:
determining the sequences of the heavy and light chain variable regions of a mouse antibody;
synthesizing a nucleic acid encoding a humanized heavy chain comprising CDRs of the mouse antibody heavy chain and a nucleic acid encoding a humanized light chain comprising CDRs of the mouse antibody light chain; and
expressing the nucleic acids in a host cell to produce a humanized antibody,
wherein the mouse antibody is 8A9.

41. A method of producing a humanized, chimeric or veneered antibody, comprising:
culturing cells transformed with nucleic acids encoding the heavy and light chains of the antibody, so that the cell secretes the antibody; and
purifying the antibody from cell culture media,
wherein the antibody is a humanized, chimeric or veneered form of 8A9.

42. A method of producing a cell line producing a humanized, chimeric or veneered antibody, comprising;
introducing a vector encoding heavy and light chains of an antibody and a selectable marker into cells;
propagating the cells under conditions to select for cells having increased copy number of the vector;
isolating single cells from the selected cells; and
banking cells cloned from a single cell selected based on yield of antibody; wherein the antibody is a humanized, chimeric or veneered form of 8A9.

43. A method of making an antibody, comprising:
obtaining a host cell of claim 39; and
maintaining the host cell under conditions in which the antibody is expressed.

44. The method of claim 43, further comprising collecting the antibody.

45. A method of testing one or more antibodies as potential therapeutics, for each test antibody the method comprising:
administering the test antibody to one or more transgenic rodents producing human IAPP (“IAPP transgenic rodents”);
determining the levels of IAPP amyloid in the one or more IAPP transgenic rodents; and
comparing IAPP amyloid levels in the IAPP transgenic rodents receiving the test antibody to blood glucose levels in control IAPP transgenic rodents that did not receive any antibody or that received a control antibody, wherein the test antibody is an anti-IAPP antibody; and
selecting the test antibody for development as a potential therapeutic if the IAPP amyloid levels in IAPP transgenic rodents receiving the test antibody are significantly lower than the IAPP amyloid levels in the control IAPP transgenic rodents.

46-47. (canceled)

48. The method of claim 45, wherein the IAPP transgenic rodent is a HIP rat.

49. The method of claim 48, wherein 10 mg/kg of test antibody is administered to each rodent weekly.

50. The method of claim 49, wherein the test antibody is administered for a period of at least 20 weeks.

51-52. (canceled)

53. The method of claim 45, further comprising administering to the control rodents a control antibody according to the same schedule as the test antibody is administered to the one or more rodents.

54. The method of claim 53, wherein the control antibody has the same isotype as the test antibody.

55. A method of reducing islet amyloid polypeptide (IAPP) accumulation in a subject having or at risk of a condition associated with IAPP accumulation, comprising administering to the subject an effective regime of an antibody defined in claim 7, thereby reducing IAPP accumulation in the subject.

56. A method of inhibiting aggregation of islet amyloid polypeptide (IAPP) in a subject having or at risk of a condition associated with IAPP accumulation, comprising administering to the subject an effective regime of an antibody defined in claim 7, thereby inhibiting aggregation of IAPP in the subject.

57. A method of stabilizing a non-toxic conformation of islet amyloid polypeptide (IAPP) in a subject having or at risk of a condition associated with IAPP accumulation, comprising administering to the subject an effective regime of an antibody defined in claim 7, thereby stabilizing a non-toxic conformation of IAPP in the subject.

58. A method of reducing islet amyloid polypeptide deposits (IAPP) in a subject having or at risk of developing IAPP deposits, comprising administering to the subject an effective regime of an antibody defined in claim 7, thereby reducing IAPP deposits in the subject.

59. A method of clearing aggregated islet amyloid polypeptide in a subject having or at risk of a condition associated with IAPP accumulation, comprising administering to the subject an effective regime of an antibody defined in claim 7, thereby clearing aggregated IAPP from the subject relative to a subject having T2D who has not received the antibody.

60. A method of reducing glucose levels in a subject having Type 2 Diabetes (T2D), comprising administering to the subject an effective regime of an antibody defined in claim 7, thereby reducing glucose levels in the subject relative to a subject having T2D who has not received the antibody.

61. A method of stabilizing glucose levels in a subject having Type 2 Diabetes (T2D), comprising administering to the subject an effective regime of an antibody defined in claim 7, thereby stabilizing glucose levels in the subject.

62. The method of claim 61, wherein the glucose levels are fasting glucose levels.

63. The method of claim 61, wherein the glucose levels are in response to an oral glucose challenge.

64. A method of increasing Beta cell numbers in a subject having or at risk of a condition associated with IAPP accumulation, comprising administering to the subject an effective regime of an antibody defined in claim 7, thereby increasing Beta cell numbers in the subject.

65. A method of stabilizing Beta cell numbers in a subject having or at risk of a condition associated with IAPP accumulation, comprising administering to the subject an effective regime of an antibody defined in claim 7, thereby increasing Beta cell numbers in the subject.

66. A method for treating or effecting prophylaxis of a condition associated with IAPP amyloid accumulation in a subject, comprising administering to the subject an effective regime of an antibody defined in claim 7.

67. The method of claim 66, wherein the condition is associated with amyloid accumulation in the pancreas of the subject.

68. The method of claim 66, wherein the condition is T2D.

69. The method of claim 66, wherein the condition is insulinoma.

70. A method for reducing inflammation associated with IAPP amyloid accumulation in a subject, comprising administering to the subject an effective amount of an antibody defined in claim 7.

71. The method of claim 70, wherein the amyloid accumulation is in the pancreas of the subject.

72. A method of reducing, ameliorating or preventing impaired glucose tolerance in a subject having or at risk of a condition associated with IAPP accumulation, comprising administering to the subject an effective regime of an antibody defined in claim 7.

73. A method of diagnosing the presence or absence of an IAPP amyloid accumulation in a pancreas of a subject, comprising contacting a sample from the subject suspected of comprising the amyloid accumulation with an effective amount of an antibody defined in claim 7.

74. The method of claim 73, further comprising detecting the binding of antibody to IAPP.

75. A method of determining a level of IAPP deposits in a subject, comprising:
administering an antibody defined in claim 7; and
detecting the presence of bound antibody in the subject.

76. The method of claim 75, wherein the presence of bound antibody is determined by positron emission tomography (PET).

77. A method for delaying the onset of T2D or insulinoma in a subject, comprising administering to the subject an effective amount of the pharmaceutical composition of claim 35.

78-80. (canceled)

81. A method of reducing beta islet cellular toxicity associated with aggregates or oligomers of IAPP, comprising administering an effective regime of the pharmaceutical composition of claim 35.

82. A method of delaying the progression in a subject from pre-diabetes to diabetes, comprising administering an effective regime of the pharmaceutical composition of claim 35.

83. A method of ameliorating impaired fasting glucose (IFG) in a subject, comprising administering an effective regime of the pharmaceutical composition of claim 35.

84. A method of ameliorating impaired glucose tolerance (IGT) in a subject, comprising administering an effective regime of the pharmaceutical composition of claim 35.

85. A method of stabilizing fasting blood glucose levels in a subject at less than 100 milligrams per deciliter after an overnight fast, comprising administering an effective regime of the pharmaceutical composition of claim 35.

86. The method of any one of claims 55-72, 77 and 81-85, wherein the subject is a human.

87. A method of reducing glucose levels in a subject having Type 1 Diabetes (T1D), comprising administering to the subject an effective regime of an antibody defined in claim 7, thereby reducing glucose levels in the subject relative to a subject having T1D who has not received the antibody.

88. A method of stabilizing glucose levels in a subject having Type 1 Diabetes (T1D), comprising administering to the subject an effective regime of an antibody defined in claim 7, thereby stabilizing glucose levels in the subject.

89. The method of claim 88, wherein the glucose levels are fasting glucose levels.

90. The method of claim 88, wherein the glucose levels are in response to an oral glucose challenge.

91. A method of reducing glucose levels in a subject having Type 1.5 Diabetes (T1.5D), comprising administering to the subject an effective regime of an antibody defined in claim 7, thereby reducing glucose levels in the subject relative to a subject having T1.5D who has not received the antibody.

92. A method of stabilizing glucose levels in a subject having Type 1.5 Diabetes (T1.5D), comprising administering to the subject an effective regime of an antibody defined in claim 7, thereby stabilizing glucose levels in the subject.

93. The method of claim 92, wherein the glucose levels are fasting glucose levels.

94. The method of claim 92, wherein the glucose levels are in response to an oral glucose challenge.

95. The antibody of claim 28, wherein the mature heavy chain variable region is fused to a heavy chain constant region having the sequence of SEQ ID NO: 42, 46, or 47 and/or the mature light chain variable region is fused to a light chain constant region having the sequence of SEQ ID NO: 44 or 49.


SEQUENCE LISTING (49 sequences)

SEQ ID NO: 1 - protein, 37 aa, Homo sapiens [sequence omitted]

SEQ ID NO: 2 - protein, 10 aa, Artificial Sequence
  Synthesized
KCNTATCATQ

SEQ ID NO: 3 - DNA, 414 bp, Artificial Sequence
  Synthesized
atggctgtcctggtgctgttcctctgcctggttgcatttccaagctgtgtcctgtcccaggtgcaattgaaggagtcaggacctggcctggtggcgccctcacagagcctgtccatcacttgcactgtgtctgggttttcattaaccaactatgctgtacactgggttcgccagcctccaggaaagggtctggagtggctgggagtaatatggtctggtggcaacacaaattataattcggctctcatgtccagactgaggatcagcaaagacaactcccagagtcaagttttcttaaagatgaacagtctgcaaactgatgacacaggcatgtactactgtgccagagatcgggggtactatggtggaatctacgggtttgctaactggggccaagggactctggtctcctca

SEQ ID NO: 4 - protein, 140 aa, Artificial Sequence
  Synthesized
MAVLVLFLCLVAFPSCVLSQVQLKESGPGLVAPSQSLSITCTVSGFSLTNYAVHWVRQPPGKGLEWLGVIWSGGNTNYNSALMSRLRISKDNSQSQVFLKMNSLQTDDTGMYYCARDRGYYGGIYGFANWGQGTLVTVSS

SEQ ID NO: 5 - DNA, 381 bp, Artificial Sequence
  Synthesized
atgagggttcctgctcacgtttttggcttcttgttgctctggtttccaggtaccagatgtgacatccagatgacccagtctccatcctccttatctgcctctctgggaaaaagagtcagtctcacttgtcgggcaagtcaggaaattagtggttacttaagctggcttcagcagaaaccagatggaactattaaacgcctgatctacgccgcatccacttcagattctggtgtcccaaaaaggttcagtggcagtaggtctgggtcagattattctctcaccatcagtagcgttgagtctgaagattttgcagactattactgtctacaatatgctgattatccgtacacgttcggaggggggaccaagctggaaataaaa

SEQ ID NO: 6 - protein, 127 aa, Artificial Sequence
  Synthesized
MRVPAHVFGFLLLWFPGTRCDIQMTQSPSSLSASLGKRVSLTCRASQEISGYLSWLQQKPDGTIKRLIYAASTSDSGVPKRFSGSRSGSDYSLTISSVESEDFADYYCLQYADYPYTFGGGTKLEIK

SEQ ID NO: 7 - protein, 121 aa, Artificial Sequence
  Synthesized
QVQLKESGPGLVAPSQSLSITCTVSGFSLTNYAVHWVRQPPGKGLEWLGVIWSGGNTNYNSALMSRLRISKDNSQSQVFLKMNSLQTDDTGMYYCARDRGYYGGIYGFANWGQGTLVTVSS

SEQ ID NO: 8 - protein, 10 aa, Artificial Sequence
  Synthesized
GFSLTNYAVH

SEQ ID NO: 9 - protein, 16 aa, Artificial Sequence
  Synthesized
VIWSGGNTNYNSALMS

SEQ ID NO: 10 - protein, 13 aa, Artificial Sequence
  Synthesized
DRGYYGGIYGFAN

SEQ ID NO: 11 - protein, 121 aa, Artificial Sequence
  Synthesized
QVQLKESGPGLVAPSQSLSITCTVSGFSLTNYGVDWVRQPPGKGLEWVGVIWSGGSTNYNSALMSRLSISKDNSKSQVFLKMNSLQTDDTAVYYCAKHWGGYYIPYGMDHWGQGTTVTVSS

SEQ ID NO: 12 - protein, 121 aa, Artificial Sequence
  Synthesized
QVQLQQSGPGLVKPSETLSLTCTVSGGSLTNYYWSWIRQPPGKGLQWIGYISHSGITNYNPSLMSRLTMSVDTPKNQFSLKLNSVTTADTGVYYCAREGVLPASIRLFDPWGQGTLVTVSS

SEQ ID NO: 13 - protein, 121 aa, Artificial Sequence
  Synthesized
QVQLQQSGPGLVKPSQSLSLTCTVSGFSLTNYAVHWVRQPPGKGLEWLGVIWSGGNTNYNSALMSRLTISKDNSQNQFSLKLNSLTTADTGVYYCARDRGYYGGIYGFANWGQGTLVTVSS

SEQ ID NO: 14 - protein, 121 aa, Artificial Sequence
  Synthesized
QVQLQQSGPGLVKPSQSLSLTCTVSGFSLTNYAVHWIRQPPGKGLEWIGVIWSGGNTNYNSALMSRLTMSVDTSKNQFSLKLNSLTTDDTGVYYCARDRGYYGGIYGFANWGQGTLVTVSS

SEQ ID NO: 15 - protein, 121 aa, Artificial Sequence
  Synthesized
QVQLQQSGPGLVKPSQSLSLTCTVSGFSLTNYAVHWVRQPPGKGLEWIGVIWSGGNTNYNSALMSRLTISKDTSKNQFSLKLNSLTTDDTGVYYCARDRGYYGGIYGFANWGQGTLVTVSS

SEQ ID NO: 16 - protein, 121 aa, Artificial Sequence
  Synthesized
QVQLQQSGPGLVKPSQSLSLTCTVSGFSLTNYAVHWVRQPPGKGLEWIGVIWSGGNTNYNSALMSRLTISKDTSKNQVFLKLNSLTTDDTGVYYCARDRGYYGGIYGFANWGQGTLVTVSS

SEQ ID NO: 17 - protein, 121 aa, Artificial Sequence
  Synthesized
QVQLQQSGPGLVKPSQSLSLTCTVSGFSLTNYAVHWIRQPPGKGLEWIGVIWSGGNTNYNSALMSRLTMSVDTSKNQVSLKLNSLTTDDTGVYYCARDRGYYGGIYGFANWGQGTLVTVSS

SEQ ID NO: 18 - protein, 121 aa, Artificial Sequence
  Synthesized
QVQLQQSGPGLVKPSQSLSLTCTVSGFSLTNYAVHWVRQPPGKGLEWIGVIWSGGNTNYNSALMSRLTISKDTSKNQVSLKLNSLTTDDTGVYYCARDRGYYGGIYGFANWGQGTLVTVSS

SEQ ID NO: 19 - protein, 121 aa, Artificial Sequence
  Synthesized
QVQLQQSGPGLVKPSQSLSLTCTVSGFSLTNYAVHWVRQPPGKGLEWLGVIWSGGNTNYNSALMSRLTISKDNSQNQVFLKLNSLTTADTGVYYCARDRGYYGGIYGFANWGQGTLVTVSS

SEQ ID NO: 20 - protein, 121 aa, Artificial Sequence
  Synthesized
EVQLQQSGPGLVKPSQSLSLTCTVSGFSLTNYAVHWVRQPPGKGLEWLGVIWSGGNTNYNSALMSRLTISKDNSQNQVFLKLNSLTTADTGVYYCARDRGYYGGIYGFANWGQGTLVTVSS

SEQ ID NO: 21 - DNA, 363 bp, Artificial Sequence
  Synthesized
caggtgcagctgcagcagtccggccccggcctggtgaagccctcccagtccctgtccctgacctgcaccgtgtccggcttctccctgaccaactacgccgtgcactgggtgcgccagccccccggcaagggcctggagtggctgggcgtgatctggtccggcggcaacaccaactacaactccgccctgatgtcccgcctgaccatctccaaggacaactcccagaaccagttctccctgaagctgaactccctgaccaccgccgacaccggcgtgtactactgcgcccgcgaccgcggctactacggcggcatctacggcttcgccaactggggccagggcaccctggtgaccgtgtcctcc

SEQ ID NO: 22 - DNA, 363 bp, Artificial Sequence
  Synthesized
caggtgcagctgcagcagtccggccccggcctggtgaagccctcccagtccctgtccctgacctgcaccgtgtccggcttctccctgaccaactacgccgtgcactggatccgccagccccccggcaagggcctggagtggatcggcgtgatctggtccggcggcaacaccaactacaactccgccctgatgtcccgcctgaccatgtccgtggacacctccaagaaccagttctccctgaagctgaactccctgaccaccgacgacaccggcgtgtactactgcgcccgcgaccgcggctactacggcggcatctacggcttcgccaactggggccagggcaccctggtgaccgtgtcctcc

SEQ ID NO: 23 - DNA, 363 bp, Artificial Sequence
  Synthesized
caggtgcagctgcagcagtccggccccggcctggtgaagccctcccagtccctgtccctgacctgcaccgtgtccggcttctccctgaccaactacgccgtgcactgggtgcgccagccccccggcaagggcctggagtggatcggcgtgatctggtccggcggcaacaccaactacaactccgccctgatgtcccgcctgaccatctccaaggacacctccaagaaccagttctccctgaagctgaactccctgaccaccgacgacaccggcgtgtactactgcgcccgcgaccgcggctactacggcggcatctacggcttcgccaactggggccagggcaccctggtgaccgtgtcctcc

SEQ ID NO: 24 - DNA, 363 bp, Artificial Sequence
  Synthesized
caggtgcagctgcagcagtccggccccggcctggtgaagccctcccagtccctgtccctgacctgcaccgtgtccggcttctccctgaccaactacgccgtgcactgggtgcgccagccccccggcaagggcctggagtggatcggcgtgatctggtccggcggcaacaccaactacaactccgccctgatgtcccgcctgaccatctccaaggacacctccaagaaccaggtcttcctgaagctgaactccctgaccaccgacgacaccggcgtgtactactgcgcccgcgaccgcggctactacggcggcatctacggcttcgccaactggggccagggcaccctggtgaccgtgtcctcc

SEQ ID NO: 25 - DNA, 363 bp, Artificial Sequence
  Synthesized
caggtgcagctgcagcagtccggccccggcctggtgaagccctcccagtccctgtccctgacctgcaccgtgtccggcttctccctgaccaactacgccgtgcactggatccgccagccccccggcaagggcctggagtggatcggcgtgatctggtccggcggcaacaccaactacaactccgccctgatgtcccgcctgaccatgtccgtggacacctccaagaaccaggtctccctgaagctgaactccctgaccaccgacgacaccggcgtgtactactgcgcccgcgaccgcggctactacggcggcatctacggcttcgccaactggggccagggcaccctggtgaccgtgtcctcc

SEQ ID NO: 26 - DNA, 363 bp, Artificial Sequence
  Synthesized
caggtgcagctgcagcagtccggccccggcctggtgaagccctcccagtccctgtccctgacctgcaccgtgtccggcttctccctgaccaactacgccgtgcactgggtgcgccagccccccggcaagggcctggagtggatcggcgtgatctggtccggcggcaacaccaactacaactccgccctgatgtcccgcctgaccatctccaaggacacctccaagaaccaggtctccctgaagctgaactccctgaccaccgacgacaccggcgtgtactactgcgcccgcgaccgcggctactacggcggcatctacggcttcgccaactggggccagggcaccctggtgaccgtgtcctcc

SEQ ID NO: 27 - DNA, 363 bp, Artificial Sequence
  Synthesized
caggtgcagctgcagcagtccggccccggcctggtgaagccctcccagtccctgtccctgacctgcaccgtgtccggcttctccctgaccaactacgccgtgcactgggtgcgccagccccccggcaagggcctggagtggctgggcgtgatctggtccggcggcaacaccaactacaactccgccctgatgtcccgcctgaccatctccaaggacaactcccagaaccaggtcttcctgaagctgaactccctgaccaccgccgacaccggcgtgtactactgcgcccgcgaccgcggctactacggcggcatctacggcttcgccaactggggccagggcaccctggtgaccgtgtcctcc

SEQ ID NO: 28 - DNA, 363 bp, Artificial Sequence
  Synthesized
gangtgcagctgcagcagtccggccccggcctggtgaagccctcccagtccctgtccctgacctgcaccgtgtccggcttctccctgaccaactacgccgtgcactgggtgcgccagccccccggcaagggcctggagtggctgggcgtgatctggtccggcggcaacaccaactacaactccgccctgatgtcccgcctgaccatctccaaggacaactcccagaaccaggtcttcctgaagctgaactccctgaccaccgccgacaccggcgtgtactactgcgcccgcgaccgcggctactacggcggcatctacggcttcgccaactggggccagggcaccctggtgaccgtgtcctcc

SEQ ID NO: 29 - protein, 107 aa, Artificial Sequence
  Synthesized
DIQMTQSPSSLSASLGKRVSLTCRASQEISGYLSWLQQKPDGTIKRLIYAASTSDSGVPKRFSGSRSGSDYSLTISSVESEDFADYYCLQYADYPYTFGGGTKLEIK

SEQ ID NO: 30 - protein, 11 aa, Artificial Sequence
  Synthesized
RASQEISGYLS

SEQ ID NO: 31 - protein, 7 aa, Artificial Sequence
  Synthesized
AASTSDS

SEQ ID NO: 32 - protein, 9 aa, Artificial Sequence
  Synthesized
LQYADYPYT

SEQ ID NO: 33 - protein, 107 aa, Artificial Sequence
  Synthesized
DIQMTQSPSSLSASLGERVSLTCRASQEISGYLSWLQQKPDGTIKRLIYAASTLDSGVPKRFSGSRSGSDYSLTISSLESEDFADYYCLQYASYPRTFGGGTKVEIK

SEQ ID NO: 34 - protein, 107 aa, Artificial Sequence
  Synthesized
DIQMTQSPSSLSASVGDRVSTICRASQSISSYLNWYQQKPGKAPKLLIYAASSLQSGVPSRFSGSGSGTDFTLTISSLQPEDFATYYCQQSYSTPLTFGGGTKVEIK

SEQ ID NO: 35 - protein, 107 aa, Artificial Sequence
  Synthesized
DIQMTQSPSSLSASVGDRVTITCRASQEISGYLSWLQQKPGKAIKRLIYAASTSDSGVPSRFSGSRSGSDYTLTISSLQPEDFATYYCLQYADYPYTFGGGTKVEIK

SEQ ID NO: 36 - protein, 107 aa, Artificial Sequence
  Synthesized
DIQMTQSPSSLSASVGDRVTITCRASQEISGYLSWYQQKPGKAPKLLIYAASTSDSGVPSRFSGSGSGTDFTLTISSLQPEDFATYYCLQYADYPYTFGGGTKVEIK

SEQ ID NO: 37 - protein, 107 aa, Artificial Sequence
  Synthesized
DIQMTQSPSSLSASVGDRVTITCRASQEISGYLSWLQQKPGKAIKRLIYAASTSDSGVPSRFSGSRSGSDYTLTISSLQPEDFADYYCLQYADYPYTFGGGTKVEIK

SEQ ID NO: 38 - DNA, 321 bp, Artificial Sequence
  Synthesized
gacatccagatgacccagtccccctcctccctgtccgcctccgtgggcgaccgcgtgaccatcacctgccgcgcctcccaggagatctccggctacctgtcctggctgcagcagaagcccggcaaggccatcaagcgcctgatctacgccgcctccacctccgactccggcgtgccctcccgcttctccggctcccgctccggctccgactacaccctgaccatctcctccctgcagcccgaggacttcgccacctactactgcctgcagtacgccgactacccctacaccttcggcggcggcaccaaggtggagatcaaa

SEQ ID NO: 39 - DNA, 321 bp, Artificial Sequence
  Synthesized
gacatccagatgacccagtccccctcctccctgtccgcctccgtgggcgaccgcgtgaccatcacctgccgcgcctcccaggagatctccggctacctgtcctggtaccagcagaagcccggcaaggcccccaagctgctgatctacgccgcctccacctccgactccggcgtgccctcccgcttctccggctccggctccggcaccgacttcaccctgaccatctcctccctgcagcccgaggacttcgccacctactactgcctgcagtacgccgactacccctacaccttcggcggcggcaccaaggtggagatcaaa

SEQ ID NO: 40 - DNA, 309 bp, Artificial Sequence
  Synthesized
gacatccagatgacccagtccccctcctccctgtccgcctccgtgggcgaccgcgtgaccatcacctgccgcgcctcccaggagatctccggctacctgtcctggctgcagcagaagcccggcaaggccatcaagcgcctgatctacgccgcctccacctccgactccggcgtgccctcccgcttctccggctcccgctccggctccgactacaccctgaccatctcctccctgcagcccgaggacttcgccgactactactgcctgcagtacgccgactacccctacaccttcggcggcggcaccaaa

SEQ ID NO: 41 - DNA, 991 bp, Artificial Sequence
  Synthesized
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagaccacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgtcaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacacgctgcccccatcccgggaggagatgaccaagaaccaggtcagcctgacctgcctggtcaaaggcttctatcccagcgacatcgcgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 42 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNVKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - DNA, 321 bp, Artificial Sequence
  Synthesized
actgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 44 - protein, 106 aa, Artificial Sequence
  Synthesized
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - DNA, 990 bp, Artificial Sequence
  Synthesized
gcctccaccaagggtccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 46 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 330 aa, Artificial Sequence
  Synthesized
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - DNA, 321 bp, Artificial Sequence
  Synthesized
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 49 - protein, 107 aa, Artificial Sequence [sequence omitted]